SEQUENCE LISTING (158 sequences)

SEQ ID NO: 1 - protein, 34 aa, Homo sapiens
EFQAALSRKVAELVHFLLLKYRAREPVTKAEMLG

SEQ ID NO: 2 - protein, 41 aa, Homo sapiens
SVVGNWQYFFPVIFSKASSSLQLVFGIELMEVDPIGHLYIF

SEQ ID NO: 3 - protein, 37 aa, Homo sapiens
YEFLWGPRALVETSYVKVLHHMVKISGGPHISYPPLH

SEQ ID NO: 4 - protein, 33 aa, Homo sapiens
MTPGTQSPFFLLLLLTVLTVVTGSGHASSTPGG

SEQ ID NO: 5 - protein, 41 aa, Homo sapiens
EMFLQIYKQGGFLGLSNIKFRPGSVVVQLTLAFREGTINVH

SEQ ID NO: 6 - protein, 38 aa, Homo sapiens
TSSNHSTSPQLSTGVSFFFLSFHISNLQFNSSLEDPST

SEQ ID NO: 7 - protein, 43 aa, Homo sapiens
PALGSTAPPVHNVTSASGSASGSASTLVHNGTSARATTTPASK

SEQ ID NO: 8 - protein, 42 aa, Homo sapiens
RAVRSLLRSHYREVLPLATFVRRLGPQGWRLVQRGDPAAFRA

SEQ ID NO: 9 - protein, 36 aa, Homo sapiens
AFTTSVRSYLPNTVTDALRGSGAWGLLLRRVGDDVL

SEQ ID NO: 10 - protein, 44 aa, Homo sapiens
KEQLRPSFLLSSLRPSLTGARRLVETIFLGSRPWMPGTPRRLPR

SEQ ID NO: 11 - protein, 41 aa, Homo sapiens
HREARPALLTSRLRFIPKPDGLRPIVNMDYVVGARTFRREK

SEQ ID NO: 12 - protein, 44 aa, Homo sapiens
RTFRREKRAERLTSRVKALFSVLNYERARRPGLLGASVLGLDDI

SEQ ID NO: 13 - protein, 41 aa, Homo sapiens
AGIRRDGLLLRLVDDFLLVTPHLTHAKTFLRTLVRGVPEYG

SEQ ID NO: 14 - protein, 38 aa, Homo sapiens
AVQWLCHQAFLLKLTRHRVTYVPLLGSLRTAQTQLSRK

SEQ ID NO: 15 - protein, 41 aa, Homo sapiens
LGSRPWMPGTPRRLPRLPQRYWQMRPLFLELLGNHAQCPYG

SEQ ID NO: 16 - protein, 37 aa, Homo sapiens
RRLVQLLRQHSSPWQVYGFVRACLRRLVPPGLWGSRH

SEQ ID NO: 17 - protein, 39 aa, Homo sapiens
HRLREEILAKFLHWLMSVYVVELLRSFFYVTETTFQKNR

SEQ ID NO: 18 - protein, 41 aa, Homo sapiens
TFQKNRLFFYRKSVWSKLQSIGIRQHLKRVQLRELSEAEVR

SEQ ID NO: 19 - protein, 46 aa, Homo sapiens
RAWRTFVLRVRAQDPPPELYFVKVDVTGAYDTIPQDRLTEVIASII

SEQ ID NO: 20 - protein, 41 aa, Homo sapiens
HGHVRKAFKSHVSTLTDLQPYMRQFVAHLQETSPLRDAVVI

SEQ ID NO: 21 - protein, 42 aa, Homo sapiens
GLLLDTRTLEVQSDYSSYARTSIRASLTFNRGFKAGRNMRRK

SEQ ID NO: 22 - protein, 42 aa, Homo sapiens
PARYEFLWGPRALAETSYVKVLEYVIKVSARVRFFFPSLREA

SEQ ID NO: 23 - protein, 42 aa, Homo sapiens
ILESLFRAVITKKVADLVGFLLLKYRAREPVTKAEMLESVIK

SEQ ID NO: 24 - protein, 38 aa, Homo sapiens
APSWPLSSSVPSQKTYQGSYGFRLGFLHSGTAKSVTCT

SEQ ID NO: 25 - protein, 34 aa, Homo sapiens
PVQLWVDSTPPPGTRVRAMAIYKQSQHMTEVVRR

SEQ ID NO: 26 - protein, 43 aa, Homo sapiens
SDGLAPPQHLIRVEGNLRVEYLDDRNTFRHSVVVPYEPPEVGS

SEQ ID NO: 27 - protein, 32 aa, Homo sapiens
MGGMNRRPILTIITLEDSSGNLLGRNSFEVRV

SEQ ID NO: 28 - protein, 40 aa, Homo sapiens
RGPESRLLEFYLAMPFATPMEAELARRSLAQDAPPLPVPG

SEQ ID NO: 29 - protein, 41 aa, Homo sapiens
LARRSLAQDAPPLPVPGVLLKEFTVSGNILTIRLTAADHRQ

SEQ ID NO: 30 - protein, 39 aa, Homo sapiens
AFLSVKKQFEELTLGEFLKLDRERAKNKIAKETNNKKKE

SEQ ID NO: 31 - protein, 41 aa, Homo sapiens
GAQYRIHTHGVFRGIQDVRRVPGVAPTLVRSASETSEKRPF

SEQ ID NO: 32 - protein, 38 aa, Homo sapiens
VQGYVLIAHNQVRQVPLQRLRIVRGTQLFEDNYALAVL

SEQ ID NO: 33 - protein, 43 aa, Homo sapiens
GGLRELQLRSLTEILKGGVLIQRNPQLCYQDTILWKDIFHKNN

SEQ ID NO: 34 - protein, 42 aa, Homo sapiens
GRILHNGAYSLTLQGLGISWLGLRSLRELGSGLALIHHNTHL

SEQ ID NO: 35 - protein, 43 aa, Homo sapiens
SGAMPNQAQMRILKETELRKVKVLGSGAFGTVYKGIWIPDGEN

SEQ ID NO: 36 - protein, 44 aa, Homo sapiens
GENVKIPVAIKVLRENTSPKANKEILDEAYVMAGVGSPYVSRKK

SEQ ID NO: 37 - protein, 40 aa, Homo sapiens
KEILDEAYVMAGVGSPYVSRLLGICLTSTVQLVTQLMPYG

SEQ ID NO: 38 - protein, 43 aa, Homo sapiens
SQDLLNWCMQIAKGMSYLEDVRLVHRDLAARNVLVKSPNHVKI

SEQ ID NO: 39 - protein, 38 aa, Homo sapiens
RLLQLHITMPFSSPMEAELVRRILSRDAAPLPRPGAVL

SEQ ID NO: 40 - protein, 35 aa, Homo sapiens
QTGTGKTLCYLMPGFIHLVLQPSLKGQRNRPGMLV

SEQ ID NO: 41 - protein, 38 aa, Homo sapiens
AEGQGTGGSTGDADGPGGPGIPDGPGGNAGGPGEAGAT

SEQ ID NO: 42 - protein, 43 aa, Homo sapiens
RPGAVLKDFTVSGNLLFMSVRDQDREGAGRMRVVGWGLGSASP

SEQ ID NO: 43 - protein, 37 aa, Homo sapiens
HGVTSAPDTRPAPGSTAPPAHGVTSAPDTRPAPGSTA

SEQ ID NO: 44 - protein, 38 aa, Homo sapiens
CSPMCKGSRCWGESSEDCQSLTRTVCAGGCARCKGPLP

SEQ ID NO: 45 - protein, 41 aa, Homo sapiens
KVPIKWMALESILRRRFTHQSDVWSYGVTVWELMTFGAKPY

SEQ ID NO: 46 - protein, 38 aa, Homo sapiens
PDGPGGNAGGPGEAGATGGRGPRGAGAARASGPGGGAP

SEQ ID NO: 47 - protein, 38 aa, Homo sapiens
TEDPGPDEAPRMPEAAPPVAPAPAAPTPAAPAPAPSWP

SEQ ID NO: 48 - protein, 33 aa, Homo sapiens
LGLSYDGLLGDNQIMPKAGLLIIVLAIIAREGD

SEQ ID NO: 49 - protein, 18 aa, Homo sapiens
RAVRSLLRSHYREVLPLA

SEQ ID NO: 50 - protein, 16 aa, Homo sapiens
RSHYREVLPLATFVRR

SEQ ID NO: 51 - protein, 20 aa, Homo sapiens
REVLPLATFVRRLGPQGWRL

SEQ ID NO: 52 - protein, 21 aa, Homo sapiens
TFVRRLGPQGWRLVQRGDPAA

SEQ ID NO: 53 - protein, 17 aa, Homo sapiens
PQGWRLVQRGDPAAFRA

SEQ ID NO: 54 - protein, 18 aa, Homo sapiens
HRLREEILAKFLHWLMSV

SEQ ID NO: 55 - protein, 21 aa, Homo sapiens
ILAKFLHWLMSVYVVELLKKK

SEQ ID NO: 56 - protein, 21 aa, Homo sapiens
HWLMSVYVVELLRSFFYVKKK

SEQ ID NO: 57 - protein, 18 aa, Homo sapiens
YVVELLRSFFYVTETTFQ

SEQ ID NO: 58 - protein, 16 aa, Homo sapiens
LRSFFYVTETTFQKNR

SEQ ID NO: 59 - protein, 17 aa, Homo sapiens
TFQKNRLFFYRKSVWSK

SEQ ID NO: 60 - protein, 18 aa, Homo sapiens
RLFFYRKSVWSKLQSIGI

SEQ ID NO: 61 - protein, 18 aa, Homo sapiens
KSVWSKLQSIGIRQHLKR

SEQ ID NO: 62 - protein, 18 aa, Homo sapiens
LQSIGIRQHLKRVQLREL

SEQ ID NO: 63 - protein, 18 aa, Homo sapiens
RQHLKRVQLRELSEAEVR

SEQ ID NO: 64 - protein, 18 aa, Homo sapiens
HREARPALLTSRLRFIPK

SEQ ID NO: 65 - protein, 19 aa, Homo sapiens
ALLTSRLRFIPKPDGLRPI

SEQ ID NO: 66 - protein, 18 aa, Homo sapiens
FIPKPDGLRPIVNMDYVV

SEQ ID NO: 67 - protein, 20 aa, Homo sapiens
LRPIVNMDYVVGARTFRREK

SEQ ID NO: 68 - protein, 19 aa, Homo sapiens
RTFRREKRAERLTSRVKAL

SEQ ID NO: 69 - protein, 17 aa, Homo sapiens
AERLTSRVKALFSVLNY

SEQ ID NO: 70 - protein, 20 aa, Homo sapiens
SRVKALFSVLNYERARRPGL

SEQ ID NO: 71 - protein, 18 aa, Homo sapiens
SVLNYERARRPGLLGASV

SEQ ID NO: 72 - protein, 17 aa, Homo sapiens
ARRPGLLGASVLGLDDI

SEQ ID NO: 73 - protein, 20 aa, Homo sapiens
AGIRRDGLLLRLVDDFLLVT

SEQ ID NO: 74 - protein, 19 aa, Homo sapiens
LLLRLVDDFLLVTPHLTHA

SEQ ID NO: 75 - protein, 18 aa, Homo sapiens
FLLVTPHLTHAKTFLRTL

SEQ ID NO: 76 - protein, 20 aa, Homo sapiens
HLTHAKTFLRTLVRGVPEYG

SEQ ID NO: 77 - protein, 18 aa, Homo sapiens
EFQAALSRKVAELVHFLL

SEQ ID NO: 78 - protein, 18 aa, Homo sapiens
SRKVAELVHFLLLKYRAR

SEQ ID NO: 79 - protein, 18 aa, Homo sapiens
LVHFLLLKYRAREPVTKA

SEQ ID NO: 80 - protein, 16 aa, Homo sapiens
LKYRAREPVTKAEMLG

SEQ ID NO: 81 - protein, 17 aa, Homo sapiens
GSVVGNWQYFFPVIFSK

SEQ ID NO: 82 - protein, 19 aa, Homo sapiens
WQYFFPVIFSKASSSLQLV

SEQ ID NO: 83 - protein, 19 aa, Homo sapiens
FSKASSSLQLVFGIELMEV

SEQ ID NO: 84 - protein, 21 aa, Homo sapiens
LQLVFGIELMEVDPIGHLYIF

SEQ ID NO: 85 - protein, 25 aa, Homo sapiens
KKKSYDGLLGDNQIMPKAGLLIKKK

SEQ ID NO: 86 - protein, 24 aa, Homo sapiens
KKKGDNQIMPKAGLLIIVLAIKKK

SEQ ID NO: 87 - protein, 22 aa, Homo sapiens
KKKAGLLIIVLAIIAREGDKKK

SEQ ID NO: 88 - protein, 18 aa, Homo sapiens
YEFLWGPRALVETSYVKV

SEQ ID NO: 89 - protein, 18 aa, Homo sapiens
PRALVETSYVKVLHHMVK

SEQ ID NO: 90 - protein, 18 aa, Homo sapiens
TSYVKVLHHMVKISGGPH

SEQ ID NO: 91 - protein, 19 aa, Homo sapiens
LHHMVKISGGPHISYPPLH

SEQ ID NO: 92 - protein, 19 aa, Homo sapiens
SAFLSVKKQFEELTLGEFL

SEQ ID NO: 93 - protein, 21 aa, Homo sapiens
KQFEELTLGEFLKLDRERAKN

SEQ ID NO: 94 - protein, 18 aa, Homo sapiens
FLKLDRERAKNKIAKETN

SEQ ID NO: 95 - protein, 18 aa, Homo sapiens
RERAKNKIAKETNNKKKE

SEQ ID NO: 96 - protein, 19 aa, Homo sapiens
GAQYRIHTHGVFRGIQDVR

SEQ ID NO: 97 - protein, 16 aa, Homo sapiens
HGVFRGIQDVRRVPGV

SEQ ID NO: 98 - protein, 18 aa, Homo sapiens
GIQDVRRVPGVAPTLVRS

SEQ ID NO: 99 - protein, 21 aa, Homo sapiens
VPGVAPTLVRSASETSEKRPF

SEQ ID NO: 100 - protein, 19 aa, Homo sapiens
RLLQLHITMPFSSPMEAEL

SEQ ID NO: 101 - protein, 20 aa, Homo sapiens
ITMPFSSPMEAELVRRILSR

SEQ ID NO: 102 - protein, 17 aa, Homo sapiens
MEAELVRRILSRDAAPL

SEQ ID NO: 103 - protein, 18 aa, Homo sapiens
RRILSRDAAPLPRPGAVL

SEQ ID NO: 104 - protein, 25 aa, Homo sapiens
KKKMTPGTQSPFFLLLLLTVLTKKK

SEQ ID NO: 105 - protein, 24 aa, Homo sapiens
KKKFFLLLLLTVLTVVTGSGHKKK

SEQ ID NO: 106 - protein, 24 aa, Homo sapiens
KKKTVLTVVTGSGHASSTPGGKKK

SEQ ID NO: 107 - protein, 20 aa, Homo sapiens
PALGSTAPPVHNVTSASGSA

SEQ ID NO: 108 - protein, 18 aa, Homo sapiens
PPVHNVTSASGSASGSAS

SEQ ID NO: 109 - protein, 18 aa, Homo sapiens
SASGSASGSASTLVHNGT

SEQ ID NO: 110 - protein, 18 aa, Homo sapiens
GSASTLVHNGTSARATTT

SEQ ID NO: 111 - protein, 18 aa, Homo sapiens
TLVHNGTSARATTTPASK

SEQ ID NO: 112 - protein, 18 aa, Homo sapiens
TSSNHSTSPQLSTGVSFF

SEQ ID NO: 113 - protein, 18 aa, Homo sapiens
TSPQLSTGVSFFFLSFHI

SEQ ID NO: 114 - protein, 19 aa, Homo sapiens
TGVSFFFLSFHISNLQFNS

SEQ ID NO: 115 - protein, 19 aa, Homo sapiens
LSFHISNLQFNSSLEDPST

SEQ ID NO: 116 - protein, 18 aa, Homo sapiens
EMFLQIYKQGGFLGLSNI

SEQ ID NO: 117 - protein, 18 aa, Homo sapiens
YKQGGFLGLSNIKFRPGS

SEQ ID NO: 118 - protein, 18 aa, Homo sapiens
LGLSNIKFRPGSVVVQLT

SEQ ID NO: 119 - protein, 18 aa, Homo sapiens
KFRPGSVVVQLTLAFREG

SEQ ID NO: 120 - protein, 17 aa, Homo sapiens
VVVQLTLAFREGTINVH

SEQ ID NO: 121 - protein, 18 aa, Homo sapiens
PDGPGGNAGGPGEAGATG

SEQ ID NO: 122 - protein, 18 aa, Homo sapiens
AGGPGEAGATGGRGPRGA

SEQ ID NO: 123 - protein, 18 aa, Homo sapiens
GATGGRGPRGAGAARASG

SEQ ID NO: 124 - protein, 17 aa, Homo sapiens
PRGAGAARASGPGGGAP

SEQ ID NO: 125 - protein, 18 aa, Homo sapiens
RGPESRLLEFYLAMPFAT

SEQ ID NO: 126 - protein, 21 aa, Homo sapiens
LLEFYLAMPFATPMEAELKKK

SEQ ID NO: 127 - protein, 20 aa, Homo sapiens
AMPFATPMEAELARRSLAQD

SEQ ID NO: 128 - protein, 20 aa, Homo sapiens
EAELARRSLAQDAPPLPVPG

SEQ ID NO: 129 - protein, 19 aa, Homo sapiens
LARRSLAQDAPPLPVPGVL

SEQ ID NO: 130 - protein, 18 aa, Homo sapiens
DAPPLPVPGVLLKEFTVS

SEQ ID NO: 131 - protein, 18 aa, Homo sapiens
PGVLLKEFTVSGNILTIR

SEQ ID NO: 132 - protein, 20 aa, Homo sapiens
EFTVSGNILTIRLTAADHRQ

SEQ ID NO: 133 - protein, 21 aa, Homo sapiens
SILESLFRAVITKKVADLVGF

SEQ ID NO: 134 - protein, 18 aa, Homo sapiens
ITKKVADLVGFLLLKYRA

SEQ ID NO: 135 - protein, 18 aa, Homo sapiens
DLVGFLLLKYRAREPVTK

SEQ ID NO: 136 - protein, 20 aa, Homo sapiens
LKYRAREPVTKAEMLESVIK

SEQ ID NO: 137 - protein, 18 aa, Homo sapiens
PARYEFLWGPRALAETSY

SEQ ID NO: 138 - protein, 18 aa, Homo sapiens
LWGPRALAETSYVKVLEY

SEQ ID NO: 139 - protein, 18 aa, Homo sapiens
LAETSYVKVLEYVIKVSA

SEQ ID NO: 140 - protein, 18 aa, Homo sapiens
VKVLEYVIKVSARVRFFF

SEQ ID NO: 141 - protein, 18 aa, Homo sapiens
VIKVSARVRFFFPSLREA

SEQ ID NO: 142 - protein, 16 aa, Homo sapiens
VQGYVLIAHNQVRQVP

SEQ ID NO: 143 - protein, 18 aa, Homo sapiens
VLIAHNQVRQVPLQRLRI

SEQ ID NO: 144 - protein, 18 aa, Homo sapiens
VRQVPLQRLRIVRGTQLF

SEQ ID NO: 145 - protein, 20 aa, Homo sapiens
RLRIVRGTQLFEDNYALAVL

SEQ ID NO: 146 - protein, 18 aa, Homo sapiens
GENVKIPVAIKVLRENTS

SEQ ID NO: 147 - protein, 18 aa, Homo sapiens
PVAIKVLRENTSPKANKE

SEQ ID NO: 148 - protein, 18 aa, Homo sapiens
LRENTSPKANKEILDEAY

SEQ ID NO: 149 - protein, 18 aa, Homo sapiens
PKANKEILDEAYVMAGVG

SEQ ID NO: 150 - protein, 21 aa, Homo sapiens
ILDEAYVMAGVGSPYVSRKKK

SEQ ID NO: 151 - protein, 18 aa, Homo sapiens
APSWPLSSSVPSQKTYQG

SEQ ID NO: 152 - protein, 18 aa, Homo sapiens
SSSVPSQKTYQGSYGFRL

SEQ ID NO: 153 - protein, 18 aa, Homo sapiens
KTYQGSYGFRLGFLHSGT

SEQ ID NO: 154 - protein, 19 aa, Homo sapiens
YGFRLGFLHSGTAKSVTCT

SEQ ID NO: 155 - protein, 18 aa, Homo sapiens
AQTGTGKTLCYLMPGFIH

SEQ ID NO: 156 - protein, 21 aa, Homo sapiens
KTLCYLMPGFIHLVLQPSKKK

SEQ ID NO: 157 - protein, 18 aa, Homo sapiens
MPGFIHLVLQPSLKGQRN

SEQ ID NO: 158 - protein, 18 aa, Homo sapiens
LVLQPSLKGQRNRPGMLV

(19) United States
(12) Patent Application Publication
(10) Pub. No.: US 2016/0199469 A1
(43) Pub. Date: Jul. 14, 2016

(54) ONCOLOGY VACCINE

(71) Applicant: ALTIMMUNE UK LIMITED, London (GB)

(72) Inventors: Bertrand V. Georges, London (GB); Carlton B. Brown, London (GB)

(21) Appl. No.: 14/916,813

(22) PCT Filed: Sep. 4, 2014
(86) PCT No.: PCT/GB2014/052675

§ 371 Date: Mar. 4, 2016

(30) Foreign Application Priority Data
Sep. 6, 2013 (GB) 1315946.2

(51) Int. Cl. A61K 39/00; A61K 45/06; C07K 14/705; A61K 39/39; C07K 14/73

(52) U.S. Cl. CPC A61K 39/0011; A61K 39/39; C07K 14/70514; C07K 14/70517; A61K 45/06

(57) ABSTRACT

A pharmaceutical composition comprising at least two peptides of from 20 to 60 amino acids in length, selected from peptides comprising a sequence of at least 20 contiguous amino acids of one of the sequences shown in SEQ ID NOs: 1 to 40 and 48 or of a sequence having at least 80% identity to one of the sequences shown in SEQ ID NOs: 1 to 40 and 48, wherein each peptide comprises at least one CD8+ T-cell epitope and/or at least one CD4+ T-cell epitope.

### FIELD OF THE INVENTION

The present invention relates to an immunogenic tumour antigen peptide-derived composition and to the treatment of cancer using the composition.

### BACKGROUND TO THE INVENTION

Cancers have accounted for 13% (7.6 million) of all deaths worldwide in 2008 and are projected to continue rising, with an estimated 13 million deaths in 2030. In spite of significant progress in recent years towards the development of new therapies, cancer is still a largely unmet medical need and the leading cause of death in industrialised countries.

The main goals of a cancer treatment programs are to cure or prolong the life of patients. Once the diagnosis and degree of spread of the tumour have been established, to the extent possible, a decision must be made regarding the most effective cancer treatment in the given socioeconomic setting. Removal of tumours can be accomplished by surgery when the tumour is localised and small in size, but the propensity of cancers to invade adjacent tissue or to spread to distant sites by microscopic metastasis often limits its effectiveness. Depending on the type and stage of cancer, chemotherapy and radiotherapy have been demonstrated to prolong life and improve the patient's quality of life but can unfortunately have a negative effect on normal cells. More targeted therapy based on small molecules such as tyrosine kinase inhibitors or monoclonal antibodies recently approved represent a type of medication aiming at blocking the growth of cancer cells by interfering with specific targeted molecules needed for carcinogenesis, and tumour growth or disease progression.

Targeted cancer therapies are expected to be more effective than current treatments and less harmful to normal cells but are also facing important limitations such as the development of resistance and the lack of tumour response in the general population.

Therapeutic vaccination against cancer aiming at promoting T-cell responses is now one of the leading targeted approaches in oncology. The main premise of cancer vaccine is stimulating the patient's immune system to attack the malignant tumour cells that are responsible for the disease. There are 2 main types of therapeutic vaccines: patient-specific (generated either from a patient's own cells or tumour); and patient-nonspecific, where a vaccine induces an immune response in vivo against specific tumour-antigens. Compared to all other standard modalities (surgery, chemotherapy, radiotherapy, and passive targeted therapy), an effective vaccine-based immune response against a tumour may be the only cancer treatment with the potential to either cure a tumour or keep it under constant restraint (i.e., immune surveillance), delaying disease progression, tumour recurrence and prolonging survival. Numerous strategies are in development in an attempt to achieve effective cancer vaccines stimulating T-cell immunity but to-date, only one therapeutic vaccine to fight prostate cancer (Provenge) has been approved by the FDA (in 2010) and EU (in 2013).

Development of therapeutic vaccines against cancer involves multiple challenges. The fundamental requirement of a therapeutic cancer is to generate a robust immune response especially a cell-mediated immune response targeting a tumour antigen in order to control and/or eradicate the tumour. While a high number of tumour antigens have been identified to date, some of which are described as immunologically active, it is now well established that most of them are too restricted in terms of expression to permit wide clinical applicability. Indeed, the expression of tumour antigens may vary between patients, tumour types, stages of the disease and treatments. Thus, no single antigen will be adequate for all tumours in a given indication. In addition, the induction of an immune response through vaccination is known to be controlled by the genetic back ground of the individual especially the Human Leukocyte Antigen (HLA) system.

Peptide-based cancer vaccines represent the most specific approach to polarise the immune system against malignant T-cells, since they are preparations made of minimal immunogenic regions of an antigen. Despite the strong rationale, the promising preclinical results and the frequent induction of antigen-specific immune responses, peptide-based cancer vaccines have yielded relatively poor results in the clinical setting.

Most of these strategies have focused on delivering short peptides corresponding to minimal T-cell epitopes. Short peptides directly bind to MHC molecules on cells that are not professional antigen-presenting cells (APC), thereby potentially inducing tolerance or anergy. Moreover, short peptides are highly restricted by HLA molecules with represent a strong limitations with regard to achieving broad population coverage.

### SUMMARY OF THE INVENTION

The present invention provides a novel cancer vaccine based on long (approximately 35-mer) peptides that encompass short minimal epitopes. These peptides are more effective immunogens than peptides consisting of the minimal epitopes. The peptides of the present invention have a tertiary structure that may protect them from exopeptidase-mediated degradation, and they are too long to be presented directly on HLA; so they must be internalized by professional APC and processed for presentation. The peptides of the invention each comprise at least one CD8+ T-cell (HLA Class I) and at least one CD4+ T-cell (HLA Class II) epitope.

Unlike short peptides, such long peptides induce memory CD8⁺ T-cell responses that are boosted dramatically on repeat vaccination in mice, and induce substantially improved tumour control compared to vaccination with short peptides. Induction of CD4+ helper T-cells reactive to epitopes within the long peptides is also necessary for long term T-cell memory. The vaccine, and preferably each peptide in the vaccine, contains epitopes that activate CD8+ and CD4+ T-cell responses in individuals with different HLA backgrounds. Thus, the vaccine of the invention has broad population coverage and induces a durable immune response against tumour antigens.

Accordingly, the present invention provides a pharmaceutical composition comprising at least two peptides of from 20 to 60 amino acids in length, selected from peptides comprising a sequence of at least 20 contiguous amino acids of a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48 or of a sequence having at least 80% identity to a sequence shown in any one of said SEQ ID NOs, wherein each peptide comprises at least one CD8+ T-cell epitope and/or at least one CD4+ T-cell epitope. Preferably, the peptide comprises at least one CD8+ T-cell epitope and/or at least one CD4+ T-cell epitope.

The composition may comprise at least one peptide comprising at least 30 amino acids of a sequence shown in any one of SEQ ID NOs: 1 to 40 and 48.

The composition preferably comprises at least one peptide that has a HLA Class II allele population coverage of: at least 60% in at least 7 geographical areas; at least 60% in at least 6 geographical areas; at least 80% in at least 5 geographical areas, at least 90% in at least 2 geographical areas and/or at least 95% in at least one geographical area and/or a HLA Class I allele population coverage of: at least 25% in at least 5 geographical areas; at least 30 in at least 2 geographical areas; and/or at least 60% in at least 1 geographical area.

The composition preferably comprises at least one peptide that that induces a specific T cell response in a healthy subject and/or a cancer patient.

At least one of the peptides may comprise a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48, or a sequence having at least 80% identity a sequence shown in any one of said SEQ ID NOs: across its whole length. One or more of the peptides may comprise one or more amino acid(s) at the N-terminus and/or C-terminus to increase the net positive charge and/or to reduce hydrophobicity of the peptide. The composition may comprise peptides derived from at least two, such as three, four or more, of MAGE3, MUC1, hTERT, MAGE1, P53, NY-ESO1, HER2/NEU, HAGE, Survivin, WT1 and LAGE1, preferably from at least two, such as three, four, or all of MAGE3, LAGE1, HAGE, NY-ESO-1 and MAGE1. Alternatively the composition may comprise peptides derived from at least two, such as three or all, of MAGE3, MUC1, hTERT and MAGE1.

The composition may preferably comprise at least one peptide comprising or consisting of a sequence as shown in Table A1. The composition may comprise 2 to 14 said peptides, and optionally at least one further peptide comprising or consisting of a sequence as shown in Table A2.

The peptides in a composition of the invention may be linked to a fluorocarbon vector. The composition may further comprise an adjuvant.

The invention provides the composition of the invention for use in the treatment or prevention of cancer, particularly for the treatment of non-small cell lung cancer, breast cancer, hepatic cancer, brain cancer, stomach cancer, pancreatic cancer, kidney cancer, ovarian cancer, myeloma, acute myelogenous leukaemia, chronic myelogenous leukaemia, head and neck cancer, colorectal cancer, renal cancer, oesophageal cancer, melanoma skin cancer and prostate cancer.

A method of treating or preventing cancer, the method comprising administering to a subject in need thereof a therapeutically effective amount of a composition according to the invention, and the use of a composition according to the invention in the manufacture of a medicament for the treatment or prevention of cancer are also provided.

In addition, the invention provides a peptide of from 20 to 60 amino acids in length comprising at least 20 contiguous amino acids of a sequence shown in any one of SED ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48 or of a sequence having at least 80% identity to a sequences shown in any one of said SEQ ID NOs, which peptide comprises at least one CD8+ T-cell epitope and/or at least one CD4+ T-cell epitope. The peptide preferably has a HLA Class II allele population coverage of: at least 60% in at least 7 geographical areas; at least 60% in at least 6 geographical areas; at least 80% in at least 5 geographical areas, at least 90% in at least 2 geographical areas and/or at least 95% in at least one geographical area and/or a HLA Class I allele population coverage of: at least 25% in at least 5 geographical areas; at least 30 in at least 2 geographical areas; and/or at least 60% in at least 1 geographical area.

The peptide is preferably recognized by T cells of a cancer patient and/or a healthy subject.

The peptide of may comprise, consist essentially or consist of the sequence shown in any one of SED ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48.

The peptide may be covalently linked to a fluorocarbon vector.

### BRIEF DESCRIPTION OF THE FIGURES

FIG. 1 shows the specific T cell responses to individual peptides measured by IFN-γ ELISpot in healthy subjects.

FIG. 2 shows the frequency of IFNγ spot forming cells (SFC) in PBMC from individuals with non-small cell lung cancer (NSCLC, n=68). Figure shows the average SFC per million PBMC in response to peptide antigens (above response in culture medium alone). *Negative control peptide. TNTC—spots too numerate to count (assigned maximum response of 6000 SFC/million).

FIG. 3 shows the frequency of IFNγ spot forming cells (SFC) in PBMC from healthy individuals (n=40). Figure shows the average SFC per million PBMC in response to peptide antigens (above response in culture medium alone). *Negative control peptide. TNTC—spots too numerate to count (assigned maximum response of 6000 SFC/million).

FIG. 4 shows the frequency of responders from NSCLC patients in the IFNγ ELISpot assay in response to peptide antigens. Responder frequency was calculated as the percentage of patients with peptide responses of greater than 500 SFC/million.

FIG. 5 shows the frequency of responders from healthy subjects in the IFNγ ELISpot assay in response peptide antigens. Responder frequency was calculated as the percentage of patients with peptide responses of greater than 500 SFC/million.

FIG. 6 shows the frequency of IFNγ spot forming cells (SFC) in PBMC from healthy individuals (n=40) or Non-Small Cell Lung Cancer (NSCLC) patients (n=68). The figure shows the median number of SFCs following stimulation the peptide antigens. Error bars represent interquartile range. Number of SFC was compared in healthy vs. NSCLC using a Mann-Whitney unpaired test, *P<0.05, **P<0.01.

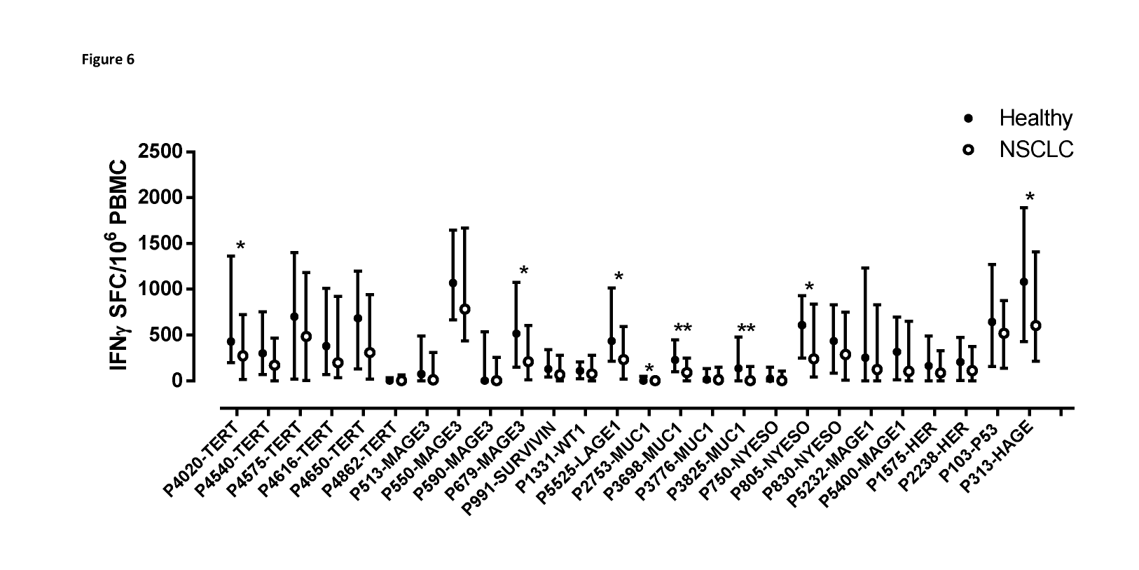

FIG. 7 shows the spot intensity for IFNγ spot forming cells (SFC) in PBMC from healthy individuals (n=40) or Non-Small Cell Lung Cancer (NSCLC) patients (n=68). The figure shows the median IFNγ spot intensity following stimulation with the peptide antigens. Spot intensity is measured as a percentage of all foreground objects per well. Error bars represent interquartile range, dotted line represents the spot intensity in wells stimulated with complete media alone (assay negative control). Spot intensity was compared in healthy vs. NSCLC using a Mann-Whitney unpaired test, *P<0.05, **P<0.01, ***P<0.001.

FIG. 8 shows CD4+ and CD8+ T cell cytokine production in PBMC from NSCLC subjects (n=7) following stimulation with peptide antigens (culture 1). Results are expressed as cytokine-producing cells, as a percentage of parent CD3/CD4 or CD3/CD8 T cell populations. The separate sections in the columns show the contribution by each individual peptide antigen in the culture.

FIG. 9 shows CD4+ and CD8+ T cell cytokine production in PBMC from NSCLC subjects (n=10) following stimulation with peptide antigens (culture 2). Results are expressed as cytokine-producing cells, as a percentage of parent CD3/CD4 or CD3/CD8 T cell populations.

FIG. 10 shows CD4+ and CD8+ T cell cytokine production in PBMC from NSCLC subjects (n=8) following stimulation with peptide antigens (culture 3). Results are expressed as cytokine-producing cells, as a percentage of parent CD3/CD4 or CD3/CD8 T cell populations. The separate sections in the columns show the contribution by each individual peptide antigen in the culture.

FIG. 11 shows CD4+ and CD8+ T cell cytokine production in PBMC from NSCLC subjects following stimulation with a single peptide antigen (culture 4 in (A), culture 5 in (B) versus a mixture of peptide antigens (culture 3 in both parts of the Figure). Results are expressed as cytokine-producing cells, as a percentage of parent CD3/CD4 or CD3/CD8 T cell populations.

### BRIEF DESCRIPTION OF THE SEQUENCE LISTING

SEQ ID NOs: 1 to 3 are the amino acid sequences of MAGE3 peptides P513, P550 and P679, respectively.

SEQ ID NOs: 4 to 7 are the amino acid sequences of MUC1 peptides P2753, P3825, P3776 and P3698, respectively.

SEQ ID NOs: 8 to 21 are the amino acid sequences of hTERT peptides P4020, P4121, P4345, P4616, P4650, P4862, P5075, P4373, P4453, P4540, P4575, P4695, P4759 and P4939, respectively.

SEQ ID NOs: 22 to 23 are the amino acid sequences of MAGE1 peptides P5400 and P5232, respectively.

SEQ ID NOs: 25 to 27 are the amino acid sequences of P53 peptides P103, P154, P205 and P262, respectively.

SEQ ID NOs: 28 and 29 are the amino acid sequences of NY-ESO-1 peptides P805 and P830, respectively.

SEQ ID NO: 30 is the amino acid sequence of Survivin peptide P991.

SEQ ID NO: 31 is the amino acid sequence of WT1 peptide P1331.

SEQ ID NOs: 32 to 38 are the amino acid sequences of HER2 peptides P1575, P1632, P1930, P2200, P2238, P2262 and P2316, respectively.

SEQ ID NO: 39 is the amino acid sequence of a LAGE1 peptide, P5525.

SEQ ID NO: 40 is the amino acid sequence of a HAGE peptide, P-HAGE.

SEQ ID NOs: 41 and 42 are the amino acid sequences of LAGE peptides, P5449 and P5566, respectively.

SEQ ID NO: 43 is the amino acid sequence of the MUC1 peptide P3150.

SEQ ID NOs: 44 and 45 are the amino acid sequences of the HER peptides P1692 and P2380, respectively.

SEQ ID NO: 46 is the amino acid sequence of the NY-ESO1 peptide P750.

SEQ ID NO: 47 is the amino acid sequence of the P53 peptide P75.

SEQ ID NO: 48 is the amino acid sequence of the MAGE3 peptide, P590.

SEQ ID NOs: 49 to 158 are the amino acid sequences of peptides shown in Annex B. As described in Example 3, each pool of 3 to 5 overlapping peptides shown in Annex B corresponds to a longer peptide sequence as disclosed herein.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an antibody” includes “antibodies”, reference to “an antigen” includes two or more such antigens, reference to “a subject” includes two or more such subjects, and the like.

A “peptide” is used herein in its broadest sense to refer to a compound of two or more subunit amino acids, amino acid analogs, or other peptidomimetics. The term “peptide” thus includes short peptide sequences and also longer polypeptides and proteins. As used herein, the term “amino acid” refers to either natural and/or unnatural or synthetic amino acids, including glycine and both the D or L optical isomers, and amino acid analogs and peptidomimetics.

By “therapeutically effective amount” of a substance, it is meant that a given substance is administered to a patient suffering from a condition, in an amount sufficient to cure, alleviate or partially arrest the condition or one or more of its symptoms. Such therapeutic treatment may result in a decrease in severity of disease symptoms, or an increase in frequency or duration of symptom-free periods. Effective amounts for a given purpose and a given agent will depend on the severity of the disease or injury as well as the weight and general state of the patient. As used herein, the term “patient” typically includes any mammal, preferably a human.

All publications, patents and patent applications cited herein, whether supra or infra, are hereby incorporated by reference in their entirety.

### Peptide Composition

The present invention provides a composition comprising broadly immunogenic peptide sequences capable of eliciting multiepitopic CD4+ and CD8+ T-cell immune responses with broad applicability in terms of population coverage. The peptides may be the only active ingredients in said composition. The composition may be tailored to a particular type of tumour or may have broad tumour coverage.

The present invention provides a pharmaceutical composition comprising at least one peptide from 20 to 60 amino acids in length, wherein said peptide comprises a fragment of at least 20 contiguous amino acids of a tumour antigen. The peptide is typically selected from peptides comprising a sequence of at least 20 contiguous amino acids of one of the sequences shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48 (that is SEQ ID NOs. 1 to 40 and 48). The peptide comprises at least one CD8+ T-cell (HLA Class I) epitope and/or at least one CD4+ T-cell (HLA Class I) epitope. The peptide elicits a response, typically a T cell response, in peripheral blood mononuclear cells (PBMC) from at least one cancer patient and/or at least one healthy subject in an in vitro assay. Particularly preferred peptides are from 20 to 60 amino acids in length and comprise a sequence of at least 20 contiguous amino acids of a sequence shown in any one of SEQ ID NOs: 40, 39, 29, 23, 2, 28, 22, 24, 18, 12, 8, 17, 3 and 11 (that is the sequences of Table A1).

The composition may comprise multiple peptides having the properties defined above. The composition may be capable of eliciting an immune response in peripheral blood mononuclear cells (PBMC) from at least two individuals of different ethnicities and/or from two individuals presenting different tumour types.

### Peptide Sequences

The composition of the invention may comprise one or more peptides comprising at least 20 contiguous amino acids, such as at least 25, 29, 30, 31, 32, 33, 34 or 35 contiguous amino acids from the sequence of any one of SEQ ID NOs: 1 to 40 and 48. Preferred peptides of the invention comprise, consist essentially of or consist of the sequence of any one of SEQ ID NOs: 1 to 40 and 48. Particularly preferred peptides comprise, consist essentially of, or consist of the sequence of any one of SEQ ID NOs: 40, 39, 29, 23, 2, 28, 22, 24, 18, 12, 8, 17, 3 and 11 (that is the sequences of Table A1).

Still further peptides that may be included in compositions of the invention are peptides that comprise a sequence that comprises one or more, such as two, three or four, amino acid substitutions, additions or deletions, preferably substitutions, within one of the sequences shown in one of SEQ ID NOs: 1 to 40 and 48. One, two, three or more amino acids within the contiguous sequence may be substituted. Substitutions within the specified sequences include mutations to remove cysteine residues. For example, cysteine residues may be substituted by serine residues.

Typically such peptides will have a sequence identity of at least 80%, such as at least 85%, 90%, 95% or 98% to at least 15 or 20, such as 25, 29, 30, 31, 32, 33, 34, 35, 36, 37, 38, 39 or 40, contiguous amino acids within one of SEQ ID NOs: 1 to 40 and 48, or to the entire length of one of the sequences shown in SEQ ID NOs: 1 to 40 and 48 (for example, as determined using the BLAST program available at the National Center for Biotechnology Information (blast.ncbi.nlm.nih.gov/Blast.cgi)).

The peptides may comprise additional sequences, provided that their overall length does not exceed 60 amino acids. For example, the peptide may comprise at least 20, such as 25, 29, 30, 31, 32, 33, 34 or 35 contiguous amino acids from within one of the sequences shown in one of SEQ ID NOs: 1 to 40 and 48, and may have a length of from 20 or 25 amino acids up to 30, 31, 32, 33, 34, 35, 36, 37, 38, 39, 40, 45, 50, 55 or 60 amino acids.

Thus, the peptide typically has a length of from 20 to 60 amino acids, such as from 25 to 50 amino acids, preferably from 30 to 40 amino acids, for example, 31, 32, 33, 34, 35, 36, 37, 38, 39, 40, 41, 42, 43, 44 or 45 amino acids.

The peptide may include additional short amino acid sequences. The additional sequences may facilitate manufacture or formulation of the peptide or enhance stability of the peptide. For example, the peptide may comprise one or more additional amino acids, typically at the N-terminus and/or the C-terminus to enhance the net positive charge of the peptide and/or to reduce the hydrophobicity of the peptide. The net positive charge may be increased so that the peptide has an isoelectric point greater than or equal to 7.

In one aspect of the invention, one or more, such as two or three positively charged amino acids (arginine and/or lysine) are added to the N- and/or C-terminus of one or more of the peptides in the composition. For example, three lysine residues may be added to the N- and/or C-terminus of one or more of the peptides. Positive amino acids are typically added at the end(s) of peptides that have an overall hydrophobicity of more than 65%, a net charge of less than zero and/or include cluster of hydrophobic amino acids.

The peptide may comprise one or more epitope that is not present in a tumour antigen. One such example is the use of fusion peptides where a promiscuous T helper epitope is covalently linked (optionally via a polypeptide linker or spacer) to the consensus sequence. As an example, the promiscuous T helper epitope can be the PADRE peptide, tetanus toxoid peptide (830-843) or influenza haemagglutinin, HA(307-319).

Where the peptide is linked to a fluorocarbon, the terminus of the peptide, such as the terminus that is not conjugated to the fluorocarbon, or other attachment, can be altered, for example to promote solubility of the fluorocarbon-peptide construct via the formation of micelles. To facilitate large-scale synthesis of the construct, the N- or C-terminal amino acid residues of the peptide can be modified. When the desired peptide is particularly sensitive to cleavage by peptidases, the normal peptide bond can be replaced by a non-cleavable peptide mimetic. Such bonds and methods of synthesis are well known in the art.

The peptide may be a native peptide. The native peptide may have free or modified extremities. The peptide may be modified to increase longevity, such as half-life or persistence at the site of administration, of the peptide in vivo or to direct the peptide to antigen-presenting cells. For example, the immunogenic peptide can contain one or more non-naturally occurring amino acids and/or non-naturally occurring covalent bonds for covalently connecting adjacent amino acids. In certain embodiments, the non-standard, non-naturally occurring amino acids can also be incorporated into the immunogenic peptides provided that they do not interfere with the ability of the peptide to interact with HLA molecules and remain cross-reactive with T-cells recognizing the natural sequences. Non-natural amino acids can be used to improve peptide resistance to protease or chemical stability. Examples of non-natural amino acids include D-amino acids and cysteine modifications.

The peptide may be coupled to a carrier, such as a protein carrier or a delivery vector. Suitable delivery vectors include lipopeptides, for example fatty acyl chains such as a monopalmitoyl chain, virosomes, liposomes and cell penetrating peptides, such as penetratin and transactivator of transcription (TAT).

One or more, and preferably all, of the peptides in the composition of the invention are preferably covalently linked to a fluorocarbon vector.

### Combinations of Peptides

A composition of the invention may comprise multiple peptides. Accordingly, the composition may comprise at least two, such as at least three, four, five, six, seven, eight, nine, ten, eleven, twelve, thirteen, fourteen, or more peptides, each of which consists of, consists essentially of or comprises the amino acid sequence shown in any one of SEQ ID NOs: 1 to 40 and 48. Each said peptide may be of 20 to 60 amino acids in length and comprise a sequence of at least 20 contiguous amino acids of any one of SEQ ID NOs: 1 to 40 and 48. Additional peptides with other sequences may be present in the compositions. One or more of the peptides may be substituted with a peptide having at least 20 contiguous amino acids of the substituted peptide or with a peptide having at least 80% identity to the amino acid sequence of the substituted peptide across its entire length.

The composition may comprise at least two, such as three, four, five, six, seven, eight, nine, ten, eleven, twelve, thirteen or fourteen peptides, selected from peptides comprising or consisting of the sequences shown in SEQ ID NOs: 1 to 40 and 48. One or more of the peptides comprising a sequence of any one of SEQ ID NOs: 1 to 40 and 48 may comprise two, three or four N- and/or C-terminal lysine residues in addition to said sequence of SEQ ID NOs: 1 to 40 and 48.

The composition may preferably comprise at least two, such as three, four, five, six, seven, eight, nine, ten, eleven, twelve, thirteen or fourteen of the peptidescomprising any one of the sequences shown in SEQ ID NOs: 40, 39, 29, 23, 2, 28, 22, 24, 18, 12, 8, 17, 3 and 11 (that is the sequences as shown in Table A1), or at least two, such as three, four, five, six, seven, eight, nine, ten, eleven, twelve or thirteen of the peptides comprising any one of the sequences shown in SEQ ID NOs: 1, 3, 5, 6, 17, 18, 22, 23, 24, 28, 32, 36 and 40.

In a composition of the invention, multiple peptides may be derived from the same tumour associated antigen. In another aspect, the multiple peptides may be derived from two or more, such as 3, 4, 5, 6, 7, 8, 9, 10 or 11 different tumour antigens. A composition comprising peptides derived from multiple tumour antigens may contain one or more than one peptide derived from a single one of the tumour antigens.

For example, the composition may comprise one or more peptides of from 20 to 60 amino acids comprising at least 20 contiguous amino acids of peptides selected from one or more, such as 2, 3, 4, 5, 6, 7, 8, 9, 10 or all, of the following groups:

(i) the MAGE3 peptides shown in SEQ ID NOs: 1 to 3 and 48;

(ii) the MUC1 peptides shown in SEQ ID NOs: 4 to 7;

(iii) the hTERT peptides shown in SEQ ID NOs: 8 to 21;

(iv) the MAGE1 peptides shown in SEQ ID NOs: 22 to 23.

(v) the P53 peptides shown in SEQ ID NOs: 24 to 23;

(vi) the NY-ESO-1 peptides shown in SEQ ID NOs: 26 to 29;

(vii) the Survivin peptide shown in SEQ ID NO: 30;

(viii) the WTI peptide shown in SEQ ID NO: 31;

(ix) the HER2 peptides shown in SEQ ID NOs: 32 to 38;

(x) the LAGE1 peptide shown in SEQ ID NOs: 39; and

(xi) the HAGE peptide shown in SEQ ID NO: 40.

The composition may comprise at least one peptide of from 20 to 60 amino acids comprising a sequence of at least 20 contiguous amino acids of one of the peptides of two, three or all of groups (i) to (iv) described above. For example, the composition may comprise a peptide comprising a sequence of at least 20 contiguous amino acids of a group (i) peptide as described above and a peptide comprising at least 20 contiguous amino acids of a group (ii) peptide, a group (iii) peptide or a group (iv) peptide as described above. In another aspect, the invention may comprise peptides comprising peptides from any one of the following combinations of peptide groups: (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv) or (i), (ii), (iii) and (iv).

Alternatively the composition may comprise at least one peptide of from 20 to 60 amino acids comprising a sequence of at least 20 contiguous amino acids of one of the peptides of two, three, four or all of groups (i), (x), (xi), (vi) and (iv) described above. For example, the composition may comprise a peptide comprising a sequence of at least 20 contiguous amino acids of a group (i) peptide as described above and a peptide comprising at least 20 contiguous amino acids of a group (x) peptide, a group (xi) peptide, a group (vi) peptide or a group (iv) peptide as described above. The composition may comprise peptides from any one of the following combinations of peptide groups: (i) and (x); (i) and (xi); (i) and (vi); (i) and (iv); (x) and (xi), (x) and (vi), (x) and (iv); (xi) and (vi); (xi) and (iv); (vi) and (iv); (i), (x) and (xi); (i), (x) and (vi); (i), (x) and (iv); (i), (xi) and (vi); (i), (xi) and (iv); (i), (vi) and (iv); (x), (xi) and (vi); (x), (xi) and (iv); (x), (vi) and (iv); (xi), (vi) and (iv); (i), (x), (xi) and (vi); (i), (x), (xi) and (iv); (i), (x), (vi) and (iv); (i), (xi), (vi) and (iv); (x), (xi), (vi) and (iv); or (i), (x), (xi), (vi) and (iv).

In addition or alternatively to considerations of tumour antigen identity, the peptides in a composition may be selected based on an assessment of their functional properties. For example, each of the peptides in the composition is preferably capable of inducing a peptide specific response in T cells of a cancer patient, such as a non-small-cell lung cancer (NSCLC) patient, and/or is capable of inducing a peptide specific response in T cells of a healthy subject. Said response in the T cells of a cancer patient is preferably of a reduced magnitude compared to said response to the same peptide in T cells of an age-matched healthy subject.

Preferably each of the peptides in a composition of the invention individually induce(s) a T cell response in at least 20% of the members of a population of cancer patients, such as a population of NSCLC patients, or in a population of healthy subjects. Such peptides may be described herein as “high responding”. The composition may comprise one, two, three, four, five, six, seven, eight, nine, ten, eleven, twelve, thirteen or fourteen peptides as active ingredients, each of which peptides induces a peptide specific T cell response in at least 20% of the members of a population of cancer patients and/or a population of healthy subjects. Preferably said peptides each induce a response in the T cells of a cancer patient that is of a reduced magnitude compared to the response to the same peptide in T cells of an age-matched healthy subject. An example of such a peptide includes a peptide comprising or consisting of the sequence of any one of SEQ ID NOs: 40, 39, 29, 23, 2, 28, 22, 24, 18, 12, 8, 17, 3 and 11. That is the sequences shown in Table A1.

Compositions comprising at least two peptides that each independently comprise or consist of a different sequence as shown in Table A1 may preferably exhibit a synergistic increase in T cell responses as compared to compositions comprising a single said peptide. Said compositions may preferably include a peptide comprising or consisting of the sequence of SEQ ID NO: 2 and a peptide comprising or consisting of the sequence of SEQ ID NO: 28, and optionally at least one additional peptide comprising or consisting of another sequence as shown in Table A1. The additional peptide preferably comprises or consists of the sequence of SEQ ID NO: 22. An example of a composition which exhibits such synergy comprises a peptide comprising or consisting of the sequence of each of SEQ ID NOs: 2, 28, 22, 3, 12, 18, 17 and 8. Without wishing to be bound by any hypothesis, the inventors consider that said synergy may result from the combination of multiple “high responding” peptides comprising sequences from different tumour antigens. Alternatively, or in addition, the peptides in such combinations do not compete with each other, e.g. for WIC binding or for presentation to T cells, and may induce “helper” effects that effectively multiply the overall response to the combination, likely through the production of cytokines, chemokines, and co-stimulatory factors.

The composition may optionally additionally include one, two, three, four, five or six peptides that individually induce(s) a peptide specific T cell response in between 10 and 20% of the members of a population of cancer patients, such as a population of NSCLC patients, and/or a population of healthy subjects. Said additional peptide or peptides may preferably be included if they individually induce a response in the T cells of a cancer patient that is of a reduced magnitude compared to the response to the same peptide in T cells of an age-matched healthy subject. An example of such a peptide includes a peptide comprising or consisting of a sequence as shown in Table A2. Preferred examples include a peptide comprising or consisting of a sequence as shown in any one of SEQ ID NOs: 1, 48, 30 and 36.

A population of cancer patients as referred to above preferably comprises at least 10 patients, more preferably 10 to 50 patients or more. Similarly, a population of healthy subjects preferably comprises at least 10 subjects, more preferably 10 to 50 subjects or more. The patients or subjects in said populations may be randomly selected from the general population and may be of any ethnicity, but are typically Caucasian.

Whether or not a peptide is able to induce a peptide specific response in T cells of a cancer patient or a healthy subject may be determined by any suitable means, typically by testing a sample of peripheral blood mononucleated cells (PBMCs) taken from said patient or subject in a suitable assay. The T cell response is thus detected in vitro in said sample. Suitable assays may measure or detect the activation of T cells following incubation with a test peptide. Activation of T cells may typically be indicated by the secretion of a cytokine, such as IFN-gamma, which may be detected in any suitable assay, typically an immunoassay such as an ELISA or ELISPOT. The magnitude of the T cell response of a patient or subject may be determined in the same assay, for example by quantifying the amount of cytokine released in the sample as a whole, or by a particular cell in the sample, following incubation with a test peptide. Suitable assays are described further below and in the Examples.

Based on the above criteria, a preferred composition of the invention comprises at least two peptides independently selected from the peptides which comprise, consist essentially of, or consist of one of any one of SEQ ID NOs: SEQ ID NOs: 40, 39, 29, 23, 2, 28, 22, 24, 18, 12, 8, 17, 3 and 11. The composition may comprise three, four, five, six, seven, eight, nine, ten, eleven, twelve, thirteen or fourteen such peptides.

A particularly preferred composition of the invention comprises:
(a) a peptide which consists of the sequence of SEQ ID NO: 40, or a peptide which is 20 to 60 amino acids in length and comprises a sequence of at least 20 contiguous amino acids of SEQ ID NO: 40;
(b) a peptide which consists of the sequence of SEQ ID NO: 39, or a peptide which is 20 to 60 amino acids in length and comprises a sequence of at least 20 contiguous amino acids of SEQ ID NO: 39;
(c) a peptide which consists of the sequence of SEQ ID NO: 29, or a peptide which is 20 to 60 amino acids in length and comprises a sequence of at least 20 contiguous amino acids of SEQ ID NO: 29;
(d) a peptide which consists of the sequence of SEQ ID NO: 23, or a peptide which is 20 to 60 amino acids in length and comprises a sequence of at least 20 contiguous amino acids of SEQ ID NO: 23; and
(e) a peptide which consists of the sequence of SEQ ID NO: 2, or a peptide which is 20 to 60 amino acids in length and comprises a sequence of at least 20 contiguous amino acids of SEQ ID NO: 2.

In said composition, the peptides of options (a) to (e) may optionally be the only active ingredients, or may optionally be the only peptide active ingredients. Alternatively, said composition may optionally further comprise any one, two, three, four or five peptides independently selected from:
(f) a peptide which consists of the sequence of SEQ ID NO: 28, or which is 20 to 60 amino acids in length and comprises a sequence of at least 20 contiguous amino acids of SEQ ID NO: 28;
(g) a peptide which consists of the sequence of SEQ ID NO: 22, or which is 20 to 60 amino acids in length and comprises a sequence of at least 20 contiguous amino acids of SEQ ID NO: 22;
(h) a peptide which consists of the sequence of SEQ ID NO: 24, or which is 20 to 60 amino acids in length and comprises a sequence of at least 20 contiguous amino acids of SEQ ID NO: 24;
(i) a peptide which consists of the sequence of SEQ ID NO: 18, or which is 20 to 60 amino acids in length and comprises a sequence of at least 20 contiguous amino acids of SEQ ID NO: 18; and
(j) a peptide which consists of the sequence of SEQ ID NO: 12, or which is 20 to 60 amino acids in length and comprises a sequence of at least 20 contiguous amino acids of SEQ ID NO: 12.

A further preferred composition of the invention comprises a peptide independently selected from each of options (a) to (g). A further preferred composition of the invention comprises a peptide independently selected from each of options (a) to (j). In each of said compositions, the peptides of options (a) to (g) or (a) to (j), respectively, may optionally be the only active ingredients, or may optionally be the only peptide active ingredients.

### Tumour Types

The combination of peptide sequences in the composition provides epitopes, preferably both CD8+ and CD4+ epitopes, present in tumour antigens from one or more type of tumour. Typically, any tumour expressing a tumour antigen corresponding to a peptide present in the composition may be treated using the composition.

Thus, the present invention provides a composition capable of eliciting an immune response in PBMC to one, two, three or more types of tumour. Each tumour expresses at least one, preferably two, three or more, such as all, of the tumour antigens present in the composition. The tumour antigens may be expressed simultaneously in the tumour or may be expressed at different times in the evolution of the tumour. The tumour antigens may be expressed in all or some of the cells of the tumour. If the tumour contains cells expressing heterogeneous tumour antigens, the composition preferably comprises at least one peptide derived from a tumour antigen expressed in each cell type.

The ability of the composition to elicit an immune response may be determined by any suitable method, such as a method described in the Examples herein. Preferably the response can be detected in PBMC from different individuals. Typically the individuals have of different HLA backgrounds, preferably at least two, preferably 3 or 4, different HLA backgrounds. The individuals of different HLA backgrounds may be of different ethnicities.

The composition of the invention preferably comprises a peptide that induces a specific T cell response in at least 20% of healthy subjects and/or cancer patients.

Immunological assays for measuring peptide-specific T cell responses in human peripheral blood mononuclear cells (PBMCs) from healthy subjects or cancer patients may be carried out by mean of cytokine ELISpot, such as the IFN-γ ELIspot assay or intracellular cytokine staining using flow cytometry.

Where the ELISpot assay is used to monitor the T cell response, a peptide of the invention or a composition of the invention may induce a specific T cell response in 20 or more cells per million PBMC, such as at least 30, 40, 50, 60, 70 or 80 cells per million PBMC, in at least 20%, such as 30% or 40% or preferably 50%, of healthy subjects and/or cancer patients.

The assays may be performed either from fresh or frozen PBMCs. The assays may be performed either ex vivo or after short term in vitro cultures of PBMCs incubated with a single peptide or a composition comprising several antigenic peptides.

The amount of the peptide(s) in the short term in vitro culture may vary from 0.001 μg per peptide to 100 μg/peptide.

The incubation time for short term in vitro cultures may be between 5 to 15 days, such as 7 to 13 days or 9 to 11 days. Short term in vitro cultures may be performed in the presence of cytokines, such as one or more of IL-2, IL-15 and IL-7, preferably IL-2 and IL-15.

Short term in vitro cultures can be performed after depletion of T regulatory cells and/or NK cells. Depletion of such cells may be particularly desirable when the PBMC are from a cancer patient. Short term in vitro culture may be performed in the presence of IL-10 neutralizing antibodies, anti-PD1 antibodies, anti-CTLA-4 antibodies, anti-OX-40 antibodies, anti-GITR antibodies, denileukin, diftitox, kinase inhibitors and/or toll receptor agonists.

### Population Coverage

The composition has a broad population coverage. The peptides in a composition of the invention comprise multiple CD8+ and CD4+ T cell epitopes that are capable of binding to different HLA alleles, so that epitopes from the tumour antigens represented by the peptides are presented on HLA molecules in a high proportion of individuals in a population.

HLA Class I and Class II molecules are polymorphic and their frequency varies between ethnic groups. Most of the polymorphism is located in the peptide-binding region, and as a result each HLA variant is believed to bind a unique repertoire of peptide ligands. HLA polymorphism represents a major challenge for vaccine designers since HLA polymorphism is the basis for differential peptide binding. Moreover, specific HLA alleles are expressed at dramatically different frequencies in different ethnicities.

Peptide binding affinity for different HLA alleles can be measured using prediction algorithm tools and/or in vitro HLA binding assays. The peptides of the invention comprise multiple HLA Class I and/or HLA Class II binding motifs. Depending on the presence of HLA allele-specific binding motifs and knowing the allelic frequencies of HLA molecules in a given population, population coverage can be calculated. Preferred peptides contains HLA Class I and HLA Class II binding motifs that can achieve an immune response in individuals with different HLA backgrounds and achieve high level of coverage in a significant number of different populations or ethnic groups.

A pharmaceutical composition of the invention typically comprises one or more peptides comprising one or more T-cell epitopes that bind to different HLA alleles to give broad population coverage. The composition may comprise peptides known or predicted to contain one or more HLA binding motif related to highly frequent HLA alleles in a specific ethnic group, or population area or across multiple ethnic groups or population areas. The composition may comprise one or more promiscuous CD4+ and CD8+ T-cell epitopes that bind to more than one allelic variant. The combination of peptide sequences in the composition provides T-cell epitopes that bind to different HLA subtypes.

The composition preferably comprises a peptide or peptides from each of the tumour antigens represented in the composition, which peptide or peptides comprise epitopes that bind to HLA Class I and/or HLA Class II alleles in individuals with different HLA backgrounds, such as from individuals from one or more geographical areas, such as at least 2, 3, 4, 5, 6, 7, 8, 9 or 10 geographical areas.

The composition may comprise a peptide that has a HLA Class II allele population coverage of: (i) at least 60% in at least 7 or 8, preferably in 9 or 10 population areas; (ii) at least 70% in at least 6 or 7, preferably at least 8, 9 or 10 population areas; (iii) at least 80% in at least 5 or 6 population areas, preferably in at least 7, 8 or 9 population areas; (iv) at least 90% in at least 2, 3 or 4 population areas, preferably at least 5, 6, 7, 8 or 9 population areas; and/or (v) at least 95% in at least one population area, preferably in at least 2, 3, 4 or 5 population areas.

The composition may comprise a peptide that has a HLA Class I allele population coverage of: (i) at least 25% in at least 5, preferably in at least 6, 7, 8, 9 or 10 population areas; (ii) at least 30% 40% or 50% in at least 2, preferably in 3, 4, 5, 6, 7, 8, 9 or 10 population areas; (iii) at least 60% in at least 1, preferably at least 2, 3, 4, 5, 6, 7, 8, 9 or 10 population areas; (iv) at least 70% in at least 1 population area, preferably in at least 2, 3, 4, 5, 6, 7, 8 or 9 population areas; (v) at least 80% in at least one population areas, preferably at least 2, 3, 4, 5, 6 or 7 population areas; and/or (vi) at least 90% in at least one population area, preferably in at least 2, 3 or 4 population areas.

The peptide may meet one, or preferably both, of the Class II allele population coverage and Class I allele population coverage criteria defined above.

Preferred peptides have a HLA Class II allele population coverage of: (i) at least 60% in at least 7 or 8, preferably in 9 or 10 population areas and a HLA Class I allele population coverage of: (i) at least 25% in at least 5, preferably in at least 6, 7, 8, 9 or 10 population areas.

Other preferred peptides have a HLA Class II allele population coverage of: (i) at least 50% in at least 9 population areas and a HLA Class I allele population coverage of: (i) at least 25% in at least 7 population areas.

The population areas are geographical areas that may be selected from Australia, Europe, North Africa, North America, North-East Asia, Oceania, South America,

South-East Asia, South-West Asia, Sub-Saharan Africa and Other. These population areas are further defined in the Examples. Population coverage may be determined as described in the Examples. The composition preferably comprises two or more peptides meeting these population coverage criteria, wherein the peptides are from two or more tumour antigens.

In one aspect, the composition of the invention elicits a response in vitro in peripheral blood mononuclear cells (PBMC) from at least two individuals with different HLA subtypes. The composition may elicit an immune response in one individual from 2, 3, 4 or more of the population areas defined above and/or in 2, 3, 4 or more individuals from different ethnic groups within one of the areas defined above.

The invention provides a composition capable of eliciting an immune response in individuals of at least two, such as three or more different ethnicities. This can be assessed using an in vitro PBMC assay as described in the Examples. The composition of the invention may be capable of eliciting an immune response in PBMC from two, three or all of: an Oriental or Indian cancer patient, a Caucasian cancer patient and an African or Arabic cancer patient.

### Fluorocarbon

The fluorocarbon can comprise one or more chains derived from perfluorocarbon or mixed fluorocarbon/hydrocarbon radicals, and may be saturated or unsaturated, each chain having from 3 to 30 carbon atoms. Thus, the chains in the fluorocarbon attachment are typically saturated or unsaturated, preferably saturated. The chains in the fluorocarbon attachment may be linear or branched, but preferably are linear. Each chain typically has from 3 to 30 carbon atoms, from 5 to 25 carbon atoms, or from 8 to 20 carbon atoms. In order to covalently link the fluorocarbon vector to the peptide, a reactive group, or ligand, for example —CO—, —NH—, S, O or any other suitable group is included in the vector. The use of such ligands for achieving covalent linkages is well known in the art. The reactive group may be located at any position on the fluorocarbon vector.

Coupling of the fluorocarbon vector to the peptide may be achieved through functional groups such as —OH, —SH, —COOH and —NH₂, naturally present or introduced onto any site of the peptide. Examples of such linkages include amide, hydrazone, disulphide, thioether and oxime bonds.

Optionally, a spacer element (peptidic or non-peptidic) can be incorporated to permit cleavage of the peptide from the fluorocarbon element for processing within an antigen-presenting cell and to optimise steric presentation of the peptide. The spacer can also be incorporated to assist in the synthesis of the molecule and to improve its stability and/or solubility. Examples of spacers include polyethylene glycol (PEG) or amino acids such as lysine or arginine that may be cleaved by proteolytic enzymes.

In one embodiment, the fluorocarbon-linked peptide can have the chemical structure CₘFₙ—C_{y}Hₓ—(Sp)-R or derivatives thereof, where m=3 to 30, n≦2m+1, y=0 to 15, x≦2y, (m+y)=3 to 30 and Sp is an optional chemical spacer moiety and R is an immunogenic peptide. Typically m and n satisfy the relationship 2m−1≦n≦2m+1, and preferably n=2m+1. Typically x and y satisfy the relationship 2y−2≦x≦2y, and preferably x=2y. Preferably the cₘFₙ-c_{y}Hₓ moiety is linear.

It is preferred that m is from 5 to 15, more preferably from 8 to 12. It is also preferred that y is from 0 to 8, more preferably from 0 to 6 or 0 to 4. It is preferred that the CₘFₙ—C_{y}Hₓ moiety is saturated (i.e., n=2m+1 and x=2y) and linear, and that m=8 to 12 and y=0 to 6 or 0 to 4.

In a particular example, the fluorocarbon vector is derived from 2H, 2H, 3H, 3H-perfluoroundecanoic acid of the following formula:

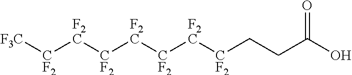 [SMILES: O=C(O)CCC(F)(F)C(F)(F)C(F)(F)C(F)(F)C(F)(F)C(F)(F)C(F)(F)C(F)(F)F]

Thus, a preferred fluorocarbon attachment is the linear saturated moiety C₈F₁₇(CH₂)₂— which is derived from C₈F₁₇(CH₂)₂COOH.

Further examples of fluorocarbon attachments have the following formulae: C₆F₁₃(CH₂)₂—, C₇F₁₅(CH₂)₂—, C₉F₁₉(CH₂)₂—, C₁₀F₂₁(CH₂)₂—, C₅F₁₁(CH₂)₃—, C₆F₁₃(CH₂)₃—, C₇F₁₅(CH₂)₃—, C₈F₁₇(CH₂)₃— and C₉F₁₉(CH₂)₃— which are derived from C₆F₁₃(CH₂)₂COOH, C₇F₁₅(CH₂)₂COOH, C₉F₁₉(CH₂)₂COOH, C₁₀F₂₁(CH₂)₂COOH, C₅F₁₁(CH₂)₃COOH, C₆F₁₃(CH₂)₃COOH, C₇F₁₅(CH₂)₃COOH, C₈F₁₇(CH₂)₃COOH and C₉F₁₉(CH₂)₃COOH respectively.

Preferred examples of suitable structures for the fluorocarbon vector-antigen constructs have the formula:

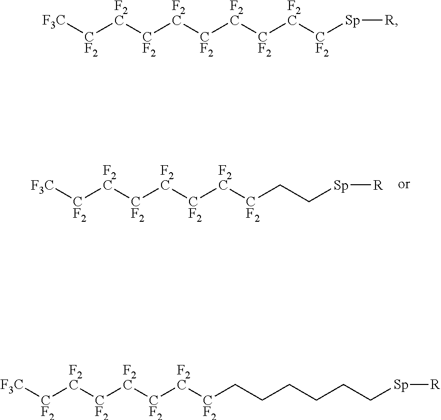

in which Sp and R are as defined above. In certain embodiments Sp is derived from a lysine residue and has the formula —CONH—(CH₂)₄—CH(NH₂)—CO—. Preferably R is any one of SEQ ID NOs: 1 to 14, preferably R is any one of SEQ ID NOs: 1 to 6. The amino group of the N-terminal amino acid of each peptide, for example, SEQ ID NO: 1, 2, 3, 4, 5 or 6, forms an amide linkage with the C-terminal carboxy group of the spacer of formula —CONH—(CH₂)₄—CH(NH₂)—CO—.

In the context of the current invention, the fluorocarbon attachment may be modified such that the resulting compound is still capable of delivering the peptide to antigen presenting cells. Thus, for example, a number of the fluorine atoms may be replaced with other halogen atoms such as chlorine, bromine or iodine. In addition, it is possible to replace a number of the fluorine atoms with methyl groups and still retain the properties of the molecule described herein.

The peptides may be linked to the fluorocarbon vector via a spacer moiety. The spacer moiety is preferably a lysine residue. This spacer residue may be present in addition to any terminal lysine residues as described above, so that the peptide may, for example, have a total of four N-terminal lysine residues. Accordingly, the preferred formulation of the invention may comprise fluorocarbon-linked peptides in which the peptides have a C-terminal or N-terminal lysine residue, preferably an N-terminal lysine residue. The terminal lysine in the peptides is preferably linked to a fluorocarbon having the formula C₈F₁₇ (CH₂)₂COOH. The fluorocarbon is preferably coupled to the epsilon chain of the N-terminal lysine residue.

It is contemplated that the pharmaceutical compositions described herein comprise at least 1, 2, 3, 4, 5, 6, 7, 8, 9 or 10 or more immunogenic peptides optionally each covalently linked to its own fluorocarbon vector.

### Peptides

The present invention also provides a peptide that is useful in a composition of the invention. The peptide may be any one of the peptides described above. In particular, the invention provides a peptide of from 20 up to 30, 35, 40, 50 or 60 amino acids in length comprising one of the sequences shown in SEQ ID NOs: 1 to 40 or a sequence that is at least 80% identical, such as at least 85%, 90%, 95% or 98% identical, to one of the sequences shown in SEQ ID NOs: 1 to 40. The peptide may include additional amino acids as described above. In one particular embodiment, the invention provides a peptide having the sequence shown in one of SEQ ID NOs: 1 to 40.

The peptide may be coupled to a carrier as described above. In one preferred aspect, the peptide of the invention is covalently linked to a fluorocarbon vector. The fluorocarbon vector may be as described above.

### Other Components

The composition of the invention may comprise an additional immunogen. The immunogen may be a B-cell antigen. The B-cell antigen can serve to stimulate an antibody response to the tumour. A pharmaceutical composition of the invention can, for example, comprise one or more fluorocarbon-linked peptides, which can stimulate a T-cell response, and a B-cell antigen.

In one aspect, the present invention provides a composition comprising two or more peptides, such as fluorocarbon-linked peptides, further comprising an adjuvant and/or optionally a pharmaceutically acceptable carrier or excipient. The excipient may be a stabiliser or bulking agent necessary for efficient lyophilisation. Examples include sorbitol, trehalose, mannitol, polyvinylpyrrolidone and mixtures thereof, preferably mannitol. Other excipients that may be present include preservatives such as antioxidants, lubricants, cryopreservatives and binders well known in the art.

An adjuvant is an agent that is able to modulate the immune response directed to a co-administered antigen while having few if any direct effects when given on its own. Such adjuvants may be capable of potentiating the immune response in terms of magnitude and/or cytokine profile. Examples of adjuvants include: natural or synthetically derived refinements of natural components of bacteria such as Freund's adjuvant & its derivatives, muramyldipeptide (MDP) derivatives, CpG, monophosphoryl lipid A; other known adjuvant or potentiating agents such as saponins, aluminium salts, cytokines, oil in water adjuvants, water-in-oil adjuvants, immunostimulating complex (ISCOMs), liposomes, formulated nano and micro-particles; bacterial toxins and toxoids; inulin, particularly gamma inulin; and TLR agonists.

Preferably, the adjuvant may be selected from the group consisting of: Peptidoglycan (such as TDM, MDP, muramyl dipeptide, Murabutide); alum solution (such as aluminium hydroxide, ADJUMER™ (polyphosphazene) or aluminium phosphate gel); glucans; algammulin; surfactants (such as squalane, Tween 80, Pluronic or squalene); calcium phosphate gel; bacterial toxins or toxoids (such as cholera holotoxin, cholera-toxin-A1-protein-A-D-fragment fusion protein, sub-unit B of the cholera toxin, or block copolymers); cytokine-containing liposomes; water-in-oil adjuvants (such as Freund's complete adjuvant, Freund's incomplete adjuvant or Montanide such as ISA 51 or ISA 720); oil-in-water adjuvants (such as MF-59); inulin-based adjuvants; cytokines (such as interferon-gamma; interleukin-1beta; interleukin-2; interleukin-7 or interleukin-12); ISCOMs (such as iscomatrix0); microspheres and microparticles of any composition; and Toll-like receptor agonists (such as CpG, ligands of human TLR 1-10, ligands of murine TLR 1-13, ISS-1018, IC31, Imidazoquinolines, Poly(I:C), Hiltonol, Ampligen, Monophosphoryl lipid A, Ribi529, cholera toxin, heat-labile toxin, Pam3Cys, CAF01 or Flagellin).

### Preparation of Pharmaceutical Compositions

The pharmaceutical compositions of the invention can be prepared by solubilising at least one peptide, such as a fluorocarbon-linked peptide, in acetic acid or in other solvents as a first step in formulating a pharmaceutical product. Examples of other solvents that may be used to disperse one or more of the fluorocarbon-linked peptides in the blend include phosphate buffered saline (PBS), propan-2-ol, tert-butanol, acetone and other organic solvents. Approaches for solubilising fluorocarbon vector-peptide conjugates are described in WO2012/090002.

The peptide or fluorocarbon-linked peptide used as a starting material is typically desiccated. Peptides and fluorocarbon-linked peptides that comprise peptides shorter than 20 amino acids and/or that have fewer than 50% hydrophobic residues can be solubilised in a solvent other than acetic acid. Acetic acid is typically used where the peptide has more than 20 amino acids and/or has more than 50% hydrophobic residues.

The concentration of fluorocarbon-linked peptide in the solution typically is from about 0.1 mM to about 10 mM, such as about 0.5 mM, 1 mM, 2 mM, 2.5 mM or 5 mM. An example of a suitable concentration is about 10 mg/mL.

The input components may be blended homogenously together to the desired ratios with any aggregates dispersed, rendered sterile and presented in a suitable format for administration. Such examples could include the introduction of a vortexing and/or sonication post-blending or post-dilution stage to facilitate solubilisation. Other permutations of the manufacturing process flow could include sterile filtration being performed at an earlier stage of the process or the omission of lyophilisation to permit a liquid final presentation.

Where the different peptides or fluorocarbon-linked peptides are solubilised separately, for example in different solvents or in different concentrations of acetic acid, the solubilised peptides or fluorocarbon-linked peptides are blended to create a mixture of peptides or fluorocarbon-linked peptides.

The optional adjuvant and/or one or more pharmaceutically acceptable excipients can also be added to the solubilised peptide/fluorocarbon-linked peptide or mixture of peptides/fluorocarbon-linked peptides. Typically, the solubilised fluorocarbon-linked peptides are mixed with the excipient and/or adjuvant.

After solubilisation and blending the solution of fluorocarbon-linked peptide(s) may be diluted. For example, the blend may be diluted in water.

The solution containing the peptides or fluorocarbon-linked peptides is preferably sterilized. Sterilisation is particularly preferred where the formulation is intended for systemic use. Any suitable means of sterilisation may be used, such as UV sterilisation or filter sterilisation. Preferably, filter sterilisation is used. Sterile filtration may include a 0.45 μm filter followed by a 0.22 μm sterilizing grade filter train.

Sterilisation may be carried out before or after addition of any excipients and/or adjuvants.

The composition of the invention may be in dried, such as lyophilized, form. The composition of the invention may be an aqueous solution, for example an aqueous solution formed by dissolving a lyophilisate or other dried formulation in an aqueous medium. The aqueous solution is typically close to neutral pH.

Drying the formulation facilitates long-term storage. Any suitable drying method may be used. Lyophilisation is preferred but other suitable drying methods may be used, such as vacuum drying, spray-drying, spray freeze-drying or fluid bed drying. The drying procedure can result in the formation of an amorphous cake within which the peptides or fluorocarbon-linked peptides are incorporated.

For long-term storage, the sterile composition may be lyophilized. Lyophilisation can be achieved by freeze-drying. Freeze-drying typically includes freezing and then drying. For example, the fluorocarbon-linked peptide mixture may be frozen for 2 hours at −80° C. and freeze-dried in a freeze drying machine for 24 hours.

Pharmaceutically acceptable compositions of the invention may be solid compositions. The fluorocarbon-linked peptide composition may be obtained in a dry powder form. A cake resulting from lyophilisation can be milled into powder form. A solid composition according to the invention thus may take the form of free-flowing particles. The solid composition typically is provided as a powder in a sealed vial, ampoule or syringe. If for inhalation, the powder can be provided in a dry powder inhaler. The solid matrix can alternatively be provided as a patch. A powder may be compressed into tablet form.

The dried, for example, lyophilised peptide or fluorocarbon-linked peptide composition may be reconstituted prior to administration. As used herein, the term “reconstitution” is understood to mean dissolution of the dried vaccine product prior to use. Following drying, such as lyophilisation, the immunogenic peptide, for example, the fluorocarbon-linked peptide product, preferably is reconstituted to form an isotonic, pH neutral, homogeneous suspension. The formulation is typically reconstituted in the aqueous phase, for example by adding Water for Injection, histidine buffer solution (such as 28 mM L-histidine buffer), sodium bicarbonate, Tris-HCl or phosphate buffered saline (PBS). The reconstituted formulation is typically dispensed into sterile containers, such as vials, syringes or any other suitable format for storage or administration.

The composition may be stored in a container, such as a sterile vial or syringe, prior to use.

### Medical Uses

The invention provides the composition of the invention for use in the treatment of the human or animal body by therapy. In particular, the composition of the invention is provided for use in a method of treating or preventing cancer. The composition of the invention elicits an immune response that may also be useful in cancer. The composition of the invention is preferably for use as a therapeutic vaccine to treat individuals with cancer.

The peptides and compositions of the invention are particularly useful in treating non-small-cell lung cancer, breast cancer, hepatic cancer, brain cancer, stomach cancer, pancreatic cancer, kidney cancer, ovarian cancer, myeloma cancer, acute myelogenous leukaemia, chronic myelogenous leukaemia, head and neck cancer, colorectal cancer, renal cancer, oesophageal cancer, melanoma skin cancer and prostate cancer patients.

The invention also provides the use of the pharmaceutical composition of the invention in the manufacture of a medicament for treating or preventing cancer, particularly non-small-cell lung cancer, breast cancer, hepatic cancer, brain cancer, stomach cancer, pancreatic cancer, kidney cancer, ovarian cancer, myeloma cancer, acute myelogenous leukaemia, chronic myelogenous leukaemia, head and neck cancer, colorectal cancer, renal cancer, oesophageal cancer, melanoma skin cancer and prostate cancer.

Similarly, the invention provides a method of treating or preventing cancer infection in a subject in need thereof, said method comprising administering to said subject a prophylactic or therapeutic amount of a composition of the present invention.

The composition of the invention may be administered in combination with a second therapeutic or prophylactic agent. For example, the second agent may comprise a further immunogen (such as a globular antigen or a recombinant or naturally occurring antigen), to further stimulate an immune response, for example to stimulate a humoral immune response where the fluorocarbon-linked peptide stimulates a cellular immune response, to cancer. It is understood that the second agent can be a B-cell antigen. In a preferred embodiment, the second agent is an agent known for use in an existing cancer therapeutic treatment. The existing cancer therapeutic agent may be selected from cyclophosmamide, alkylating-like agents such as cisplatin, plant alkaloids and terpenoids such as vincristine or paclitaxel, antimetabolites such as 5-fluorouracil, topoisomerase inhibitors type I or II such as camptothecin or doxorubicin, cytotoxic antibiotics such as actinomycin or anthracyclines such as epirubicin.

The second agent may be one, or a combination of: immunotherapeutics or immunomodulators, such as TLR agonists; agents that down-regulate T-regulatory cells such as cyclophosphamide; or agents designed to block immune checkpoints in the form of cytokines or monoclonal antibodies, such as anti-PD1 and anti-CTLA-4.

Where a second therapeutic agent or prophylactic agent is used in conjunction with a composition of the invention, administration may be contemporaneous or separated by time. The composition of the invention may be administered before, together with or after the second therapeutic agent.

Compositions of the invention can be administered to a human or animal subject in vivo using a variety of known routes and techniques. For example, the composition may be provided as an injectable solution, suspension or emulsion and administered via parenteral, subcutaneous, oral, epidermal, intradermal, intramuscular, interarterial, intraperitoneal, intravenous injection using a conventional needle and syringe, or using a liquid jet injection system. The composition may be administered topically to skin or mucosal tissue, such as nasally, intratrachealy, intestinally, sublingually, rectally or vaginally, or provided as a finely divided spray suitable for respiratory or pulmonary administration. In a preferred embodiment, the compositions are administered intramuscularly. The composition may alternatively be administered directly into a tumour, for example by intra-tumoural injection.

The composition can be administered to a subject in an amount that is compatible with the dosage composition and that will be prophylactically and/or therapeutically effective. The administration of the composition of the invention may be for either “prophylactic” or “therapeutic” purpose. As used herein, the term “therapeutic” or “treatment” includes any one or more of the following: the prevention of tumourogenesis/carcinogenesis; the reduction or elimination of symptoms; and the reduction or complete elimination of a tumour or cancer.

Treatment may be effected prophylactically (prior to confirmed diagnosis of the cancer) or therapeutically (following diagnosis of the cancer). Therapeutic treatment may be given to Stage I, II, III, or IV cancers, pre- or post-surgical intervention. The treatment may be post-surgery maintenance treatment or a long-term treatment to improve progression free survival or overall survival and/or clearance of disease.

The choice of carrier, if required, is frequently a function of the route of delivery of the composition. Within this invention, compositions may be formulated for any suitable route and means of administration. Pharmaceutically acceptable carriers or diluents include those used in compositions suitable for oral, ocular, rectal, nasal, topical (including buccal and sublingual), vaginal or parenteral (including subcutaneous, intramuscular, intravenous, intradermal, transdermal) administration.

The composition may be administered in any suitable form, for example as a liquid, solid or aerosol. For example, oral formulations may take the form of emulsions, syrups or solutions or tablets or capsules, which may be enterically coated to protect the active component from degradation in the stomach. Nasal formulations may be sprays or solutions. Transdermal formulations can be adapted for their particular delivery system and may comprise patches. Formulations for injection may be solutions or suspensions in distilled water or another pharmaceutically acceptable solvent or suspending agent.

The appropriate dosage of the prophylactic or therapeutic vaccine to be administered to a patient will be determined in the clinic. However, as a guide, a suitable human dose, which may be dependent upon the preferred route of administration, may be from 1 to 1000 μg, such as about 100 μg, 200 μg, 500 μg or 1000 μg. Multiple doses may be required to achieve an immunological or clinical effect, which, if required, will be typically administered between 1 to 12 weeks apart. Where boosting of the immune response over longer periods is required, repeat doses 1 month to 5 years apart may be applied.

The following Examples illustrate the invention.

### Example 1

### Population Coverage of Tumour Antigens

HLA Class I and II epitope prediction and calculation of population coverage was performed on 47 peptide sequences (SEQ ID NOs: 1 to 47) derived from MAGE-3, MUC1, hTERT, MAGE-1, P53, NY-ESO1, HER2/NEU, HAGE, Survivin, WT1 and LAGE1. The peptide sequences are shown in Annex A below, which also includes the sequence of SEQ ID NO: 48.

The prediction of HLA Class I peptide ligands was performed using two epitope prediction methods available at www.IEDB.org and used on 14 Aug. 2013: (1) an artificial neural network-based method (ANN; Nielsen M, Lundegaard C, Worning P, Lauemøller SL, Lamberth K, Buus S, Brunak S, Lund O. Reliable prediction of T-cell epitopes using neural networks with novel sequence representations. Protein Sci. 2003 May; 12(5):1007-17) and (2) a Stabilized Matrix-based Method (SMM, Peters B, Sette A. Generating quantitative models describing the sequence specificity of biological processes with the stabilized matrix method. BMC Bioinformatics. 2005 May 31; 6:132). HLA Class I alleles considered for the analysis were: HLA-A*01:01, HLA-A*02:01, HLA-A*02:03, HLA-A*03:01, HLA-A*11:01, HLA-A*23:01, HLA-A*24:02, HLA-A*26:01, HLA-A*29:02, HLA-A*29:02, HLA-A*30:01, HLA-A*30:01, HLA-A*30:02, HLA-A*31:01, HLA-A*32:01, HLA-A*33:03, HLA-A*68:02, HLA-B*57:01, HLA-B*07:02, HLA-B*08:01, HLA-B*15:01, HLA-B*15:02, HLA-B*15:03, HLA-B*18:01, HLA-B*27:05, HLA-B*35:01, HLA-B*39:01, HLA-B*40:01, HLA-B*40:02, HLA-B*44:02, HLA-B*44:03, HLA-B*45:01, HLA-B*46:01, HLA-B*48:01, HLA-B*51:01, HLA-B*53:01 and HLA-B*58:01. Prediction analyses were limited to potential epitopes derived from the long peptide sequences having a length of 9 amino acids and 10 amino acids for each allele.

The predicted output is given in units of IC₅₀ nM. It is well established that peptides with IC₅₀ values <50 nM are considered high affinity, between 50 and 500 nM intermediate affinity and between 500 and 5000 nM low affinity. In this analysis, peptides having a predicted affinity <50 nM using either the ANN method or the SMM method where considered as binder.

The prediction of HLA Class II peptide ligands was performed using the method developed by Sturniolo et al. (Sturniolo T, Bono E, Ding J, Raddrizzani L, Tuereci O, Sahin U, Braxenthaler M, Gallazzi F, Protti M P, Sinigaglia F, Hammer J. Generation of tissue-specific and promiscuous HLA ligand databases using DNA microarrays and virtual HLA Class II matrices. Nat Biotechnol. 1999 June; 17(6):555-61) available at www.IEDB.org and used on 14 Aug. 2013. HLA Class II alleles considered for the analysis were: HLA-DRA*01:01/HLA-DRB1*01:01, DRA*01:01/HLA-DRB1*03:01, DRA*01:01/HLA-DRB1*04:01, DRA*01:01/HLA-DRB1*07:01, DRA*01:01/HLA-DRB1*08:02, DRA*01:01/HLA-DRB1*11:01, DRA*01:01/HLA-DRB1*13:01 and DRA*01:01/HLA-DRB1*15:01 respectively considered as representative members of HLA-DR1, HLA-DR3, HLA-DR4, HLA-DR7, HLA-DR8, HLA-DR11, HLA-DR13 and HLA-DR15 antigen groups. Peptides with a score>1 are considered high affinity. For example, the high affinity universal T helper epitope HA(307-319) has a binding score of 6.12, 4.5, 3, 2.86, 2.66, 2.06, 1.8 and 1.6 for HLA-DRB1*07:01, HLA-DRB1*04:01, HLA-DRB1*11:01, HLA-DRB1*13:01, HLA-DRB1*15:01, HLA-DRB1*03:01, HLA-DRB1*01:01 and HLA-DRB1*08:02 respectively. The other high affinity universal T helper epitopes TT(830-844) has a binding score of 5.6, 3.5, 2.5, 2.1, 1.6, 1, 0.8, 0.6 for HLA-DRB1*07:01, HLA-DRB1*13:01, HLA-DRB1*03:01, HLA-DRB1*04:01, HLA-DRB1*15:01, HLA-DRB1*01:01, HLA-DRB1*08:02, HLA-DRB1*11:01 respectively. The high affinity promiscuous peptide CLIP(81-104) from the invariant chain peptide has a binding score of 6.3, 5.6, 5.4, 5.38, 4.2, 2.9, 2.78, 2.4 for HLA-DRB1*07:01, HLA-DRB1*13:01, HLA-DRB1*03:01, HLA-DRB1*15:01, HLA-DRB1*11:01, HLA-DRB1*04:01, HLA-DRB1*01:01 and HLA-DRB1*08:02 respectively.

Based on the presence of predicted epitopes for specific HLA molecules and or groups of epitopes in a given peptide, population coverage for a specific peptide containing epitopes was calculated using the resource analysis available at www.IEDB.org, used on 14 Aug. 2013. The selected principal population areas (as defined at www.IEDB.org) include Australia (corresponding to Cape York, Groote Eylandt, Kimberley and Yuendumu populations), Europe (corresponding to Bulgarian, Croatian, Cuban-white, Czech, Finn 90, Georgian, Irish, North America-Caucasian and Slovenian populations), North Africa (corresponding to Algerian 99, Chaouya, Metalsa, Moroccan 98 and Moroccan 99 populations), North America (corresponding to Amerindian, Lacandon, Seri and Yupik populations), North-East Asia (corresponding to Buriat, Korean 200 and Tuva populations), Oceania (corresponding to American Samoa, Filipino, Ivatan populations), Other (corresponding to Brazilian-Admixed, African and European, Cuban-Mulatto, Mexican and North America-Hispanic populations), South America (corresponding to Bari, Guarani-Kaiowa and Guarani-Nandewa populations), South-East Asia (corresponding to Ami 97, Atayal, Bunun, Chinese, Hakka, Han-Chinese 149, Han-Chinese 572, Kinh, Malay, Minnan, Muong, North America-Asian pacific islander, Okinawan, Paiwan 51, Pazeh, Puyuma 49, Rukai, Ryukuan, Saisiat, Singapore-Chinese, Siraya, Thai, Thao, Toroko, Tsou and Yami populations), South-West Asia (corresponding to Arab Druze, Israeli Jews, Kurdish, Omani and Turk populations) and Sub-Saharan Africa (corresponding to Doggon, Kenyan 142, Kenyan Highlander, Kenyan Lowlander, Mandenka, North America-African, Rwandan, Shona, Ugandan, Zambian and Zulu populations).

For the purpose of calculating population coverage, due to the limited number of HLA Class II alleles used in the epitope prediction, groups of HLA class II alleles were defined. The definition of groups of HLA class II alleles relies on the high degree of promiscuous peptide binding across HLA Class II alleles belonging to the same HLA group (Wilson C C, Palmer B, Southwood S, Sidney J, Higashimoto Y, Appella E, Chesnut R, Sette A, Livingston BD. Identification and antigenicity of broadly cross-reactive and conserved human immunodeficiency virus type 1-derived helper T-lymphocyte epitopes. J Virol. 2001 May; 75(9):4195-207; Lund O, Nielsen M, Kesmir C, Petersen A G, Lundegaard C, Worning P, Sylvester-Hvid C, Lamberth K, Røder G, Justesen S, Buus S, Brunak S. Definition of supertypes for HLA molecules using clustering of specificity matrices. Immunogenetics. 2004 March; 55 (12):797-810). Groups of HLA Class II alleles (representative of the molecules used in the epitope prediction) were defined as follows: HLA-DR1 (HLA DRB1*01, HLA DRB1*0101, HLA DRB1*0102, HLA DRB1*010201, HLA DRB1*0103), HLA-DR3 (HLA DRB1*03, HLA DRB1*0301, HLA DRB1*030101, HLA DRB1*0302, HLA DRB1*030201, HLA DRB1*0303, HLA DRB1*0305, HLA DRB1*0308, HLA DRB1*0317), HLA-DR4 (HLA DRB1*04, HLA DRB1*0401, HLA DRB1*040101, HLA DRB1*0402, HLA DRB1*0403, HLA DRB1*040301, HLA DRB1*0404, HLA DRB1*0405, HLA DRB1*040501, HLA DRB1*0406, HLA DRB1*0407, HLA DRB1*0408, HLA DRB1*0410, HLA DRB1*0411, HLA DRB1*0412, HLA DRB1*0413, HLA DRB1*0415, HLA DRB1*0416, HLA DRB1*0436), HLA-DR7 (HLA DRB1*07, HLA DRB1*0701, HLA DRB1*070101), HLA-DR8 (HLA DRB1*08, HLA DRB1*0801, HLA DRB1*0802, HLA DRB1*080201, HLA DRB1*080302, HLA DRB1*0804, HLA DRB1*080401, HLA DRB1*080402, HLA DRB1*0805, HLA DRB1*0806, HLA DRB1*0807, HLA DRB1*0808, HLA DRB1*0809, HLA DRB1*0811, HLA DRB1*0818), HLA-DR11 (HLADRB1*11, HLADRB1*1101, HLADRB1*110101, HLADRB1*110102, HLA DRB1*1102, HLA DRB1*1103, HLA DRB1*1104, HLA DRB1*110401, HLADRB1*1108, HLADRB1*1109, HLADRB1*1111, HLA DRB1*1113, HLADRB1*111401, HLADRB1*111901, HLADRB1*1130), HLA-DR13 (HLA DRB1*13, HLA DRB1*1301, HLA DRB1*1302, HLADRB1*130201, HLA DRB1*1303, HLA DRB1*130301, HLA DRB1*130302, HLA DRB1*1304, HLA DRB1*1305, HLA DRB1*1306, HLA DRB1*1307, HLA DRB1*1309, HLA DRB1*1310, HLADRB1*1312, HLADRB1*1317, HLADRB1*1320, HLA DRB1*1323, HLA DRB1*1325, HLA DRB1*1327, HLADRB1*1331) and HLA-DR15 (HLA DRB1*15, HLA DRB1*1501, HLA DRB1*150101, HLA DRB1*1502, HLA DRB1*150201, HLA DRB1*1503, HLA DRB1*1504, HLA DRB1*1505, HLA DRB1*1506).

Results of population coverage calculation are presented in Table 1 for the HLA Class I alleles and in Table 2 for the HLA Class II alleles.

Based on the alleles selected in this approach, maximum population coverages for each of the population areas are: Australia (86.68%), Europe (99.69%), North Africa (83.11%), North America (98.21%), North-East Asia (83.80%), Oceania (95.18%), Other (96.71%), South America (50.67%), South-East Asia (96.03%), South-West Asia (93.06%), Sub-Saharan Africa (90.37%) for the HLA Class I alleles and Australia (89.67%), Europe (99.64%), North Africa (99.28%), North America (90.47%), North-East Asia (90.83%), Oceania (91.29%), Other (97.18%), South America (57.45%), South-East Asia (75.50%), South-West Asia (97.06%) and Sub-Saharan Africa (98.74%).

Preferred peptides with regard to their ability to achieve broad population coverage were defined as follows: Peptides having a PC %>60% in at least 7, or at least 8 different population areas or preferentially at least 9 population areas (out of the 11 population area described above) for the HLA Class II alleles and a PC %>25% in at least 2, 3, 4, 5 or 6 population areas or preferentially at least 7 population areas (out of the 11 population area described above) for the HLA Class I alleles.

For example, P2380_HER, P5566_LAGE1, P75-P53, P750-NY-ESO, P1692-HER, P3150-MUC, P5449-LAGE for example do not fall under the definition of the preferred peptides.

For example, P513_MAGE3, P550_MAGE3, P679_MAGE3, P2753_MUC1, P3776_MUC1, P4020_hTERT, P4345_hTERT, P4373_hTERT, P4540_hTERT, P4616_hTERT, P4650_hTERT, P4695_hTERT, P4759_hTERT, P4862_TERT, P4939_hTERT, P5075_hTERT, P5400_MAGE1 and P5232_MAGE1 derived from MAGE-3, MUC-1, Telomerase and MAGE-1 respectively fall under the definition of preferred peptides with each peptide having an PPC %>50% in at least 9 different population areas for the HLA Class II alleles and a PPC %>25% in at least 7 population area for the HLA Class I alleles.

TABLE 1
Population coverage (EPC %) calculated for each peptide based on HLA
Class I peptide binding predictions

| | | | North | North | North-East | | | South | South-East | South-West | Sub-Saharan |
|---|---|---|---|---|---|---|---|---|---|---|---|
| | Australia | Europe | Africa | America | Asia | Oceania | Other | America | Asia | Asia | Africa |
| P103-P53 | 50% | 87% | 34% | 64% | 54% | 60% | 75% | 42% | 63% | 69% | 65% |
| P154-P53 | 27% | 39% | 16% | 11% | 20% | 35% | 28% | 0% | 45% | 30% | 36% |
| P205-P53 | 1% | 14% | 12% | 31% | 7% | 2% | 15% | 1% | 13% | 7% | 28% |
| P262_P53 | 39% | 66% | 1% | 48% | 34% | 58% | 51% | 40% | 54% | 32% | 25% |
| P513_MAGE3 | 56% | 69% | 17% | 45% | 46% | 69% | 61% | 40% | 70% | 51% | 48% |
| P550_MAGE3 | 84% | 94% | 67% | 93% | 78% | 94% | 89% | 48% | 94% | 85% | 79% |
| P679_MAGE3 | 27% | 67% | 18% | 44% | 25% | 40% | 57% | 41% | 34% | 42% | 49% |
| P805_NY_ESO1 | 27% | 66% | 36% | 57% | 42% | 38% | 63% | 41% | 37% | 58% | 59% |
| P830_NY_ESO1 | 37% | 53% | 13% | 31% | 29% | 58% | 48% | 40% | 53% | 34% | 42% |
| P991_SURVIVIN | 38% | 22% | 8% | 8% | 21% | 52% | 16% | 1% | 56% | 22% | 17% |
| P1331_WT1 | 27% | 39% | 36% | 27% | 27% | 34% | 29% | 0% | 45% | 34% | 45% |
| P1575_HER | 42% | 63% | 10% | 55% | 37% | 60% | 52% | 44% | 61% | 40% | 24% |
| P1632_HER | 41% | 48% | 8% | 26% | 27% | 62% | 31% | 1% | 68% | 30% | 26% |
| P1930_HER | 40% | 53% | 6% | 33% | 31% | 60% | 46% | 44% | 59% | 33% | 31% |
| P2200_HER | 27% | 40% | 6% | 23% | 15% | 46% | 25% | 2% | 53% | 22% | 24% |
| P2238-HER | 58% | 86% | 49% | 65% | 57% | 79% | 79% | 42% | 78% | 74% | 72% |
| P2262-HER | 25% | 65% | 39% | 57% | 38% | 48% | 67% | 41% | 43% | 59% | 62% |
| P2316_HER | 7% | 33% | 4% | 23% | 10% | 23% | 23% | 8% | 32% | 12% | 24% |
| P2380_HER | 1% | 10% | 16% | 15% | 12% | 8% | 15% | 1% | 13% | 12% | 36% |
| P2753_MUC1 | 25% | 52% | 12% | 38% | 23% | 40% | 48% | 40% | 32% | 33% | 42% |
| P3698_MUC1 | 26% | 60% | 10% | 33% | 23% | 39% | 50% | 40% | 31% | 31% | 37% |
| P3825_MUC1 | 10% | 58% | 25% | 48% | 16% | 24% | 39% | 11% | 36% | 23% | 37% |
| P3776_MUC1 | 47% | 71% | 20% | 45% | 42% | 66% | 61% | 41% | 65% | 52% | 55% |
| P4020_hTERT | 43% | 51% | 31% | 43% | 30% | 64% | 35% | 8% | 72% | 31% | 38% |
| P4121_hTERT | 3% | 35% | 16% | 32% | 13% | 19% | 26% | 4% | 25% | 13% | 28% |
| P4345_hTERT | 28% | 69% | 7% | 55% | 29% | 40% | 54% | 40% | 34% | 40% | 28% |
| P4373_hTERT | 49% | 46% | 16% | 78% | 27% | 58% | 39% | 11% | 54% | 13% | 29% |
| P4453_hTERT | 3% | 35% | 2% | 37% | 7% | 2% | 21% | 2% | 4% | 6% | 22% |
| P4540_hTERT | 61% | 96% | 72% | 73% | 72% | 81% | 88% | 42% | 83% | 87% | 85% |
| P4575_hTERT | 25% | 17% | 20% | 5% | 18% | 45% | 18% | 0% | 51% | 27% | 33% |
| P4616_hTERT | 32% | 58% | 8% | 32% | 22% | 39% | 38% | 9% | 53% | 30% | 32% |
| P4650_hTERT | 31% | 71% | 23% | 39% | 34% | 47% | 57% | 44% | 47% | 43% | 50% |
| P4695_hTERT | 25% | 52% | 15% | 38% | 23% | 40% | 49% | 40% | 33% | 35% | 42% |
| P4759_hTERT | 29% | 64% | 27% | 43% | 25% | 43% | 54% | 44% | 40% | 39% | 48% |
| P4862_TERT | 50% | 74% | 6% | 38% | 41% | 65% | 58% | 44% | 64% | 51% | 29% |
| P4939_hTERT | 49% | 82% | 35% | 50% | 54% | 65% | 73% | 40% | 66% | 71% | 62% |
| P5075_hTERT | 50% | 77% | 8% | 57% | 45% | 66% | 63% | 44% | 65% | 53% | 41% |
| P5400_MAGE1 | 63% | 96% | 57% | 65% | 66% | 79% | 83% | 45% | 82% | 79% | 78% |
| P5232_MAGE1 | 28% | 43% | 17% | 24% | 20% | 36% | 30% | 1% | 47% | 30% | 34% |
| P5525_LAGE1 | 23% | 59% | 29% | 50% | 40% | 50% | 48% | 5% | 60% | 37% | 54% |
| P5566_LAGE1 | 0% | 6% | 2% | 19% | 7% | 17% | 7% | 0% | 12% | 5% | 15% |
| P-HAGE | 29% | 79% | 9% | 61% | 36% | 42% | 65% | 42% | 37% | 46% | 48% |
| P75-P53 | 26% | 66% | 16% | 31% | 26% | 39% | 42% | 29% | 56% | 42% | 50% |
| P750-NYESO | 0% | 0% | 0% | 0% | 0% | 0% | 0% | 0% | 0% | 0% | 0% |
| P1692-HER | 3% | 5% | 4% | 14% | 4% | 3% | 2% | 1% | 8% | 59% | 10% |
| P3150-MUC | 2% | 25% | 10% | 0% | 1% | 3% | 21% | 1% | 14% | 1% | 8% |
| P5449-LAGE | 0% | 0% | 0% | 0% | 0% | 0% | 0% | 0% | 0% | 0% | 0% |

TABLE 2
Population coverage (EPC %) calculated for each peptide based on HLA
Class II peptide binding predictions

| | | | North | North | North-East | | | South | South-East | South-West | Sub-Saharan |
|---|---|---|---|---|---|---|---|---|---|---|---|
| | Australia | Europe | Africa | America | Asia | Oceania | Other | America | Asia | Asia | Africa |
| P103-P53 | 90% | 100% | 99% | 90% | 91% | 91% | 97% | 57% | 76% | 97% | 99% |
| P154-P53 | 89% | 97% | 98% | 90% | 87% | 91% | 94% | 56% | 75% | 95% | 97% |
| P205-P53 | 89% | 97% | 98% | 90% | 87% | 91% | 94% | 56% | 75% | 95% | 97% |
| P262_P53 | 89% | 97% | 98% | 90% | 87% | 91% | 94% | 56% | 75% | 95% | 97% |
| P513_MAGE3 | 76% | 87% | 90% | 35% | 73% | 85% | 76% | 40% | 61% | 87% | 96% |
| P550_MAGE3 | 58% | 99% | 99% | 83% | 85% | 88% | 92% | 27% | 64% | 96% | 96% |
| P679_MAGE3 | 90% | 100% | 99% | 90% | 91% | 91% | 97% | 57% | 76% | 97% | 99% |
| P805_NY_ESO1 | 35% | 93% | 92% | 17% | 71% | 81% | 73% | 6% | 46% | 88% | 95% |
| P830_NY_ESO1 | 89% | 95% | 94% | 85% | 84% | 87% | 89% | 55% | 64% | 82% | 87% |
| P991_SURVIVIN | 57% | 96% | 97% | 83% | 81% | 87% | 87% | 26% | 63% | 93% | 92% |
| P1331_WT1 | 90% | 100% | 99% | 90% | 91% | 91% | 97% | 57% | 76% | 97% | 99% |
| P1575_HER | 90% | 100% | 99% | 90% | 91% | 91% | 97% | 57% | 76% | 97% | 99% |
| P1632_HER | 90% | 100% | 99% | 90% | 91% | 91% | 97% | 57% | 76% | 97% | 99% |
| P1930_HER | 90% | 100% | 99% | 90% | 91% | 91% | 97% | 57% | 76% | 97% | 99% |
| P2200_HER | 90% | 100% | 99% | 90% | 91% | 91% | 97% | 57% | 76% | 97% | 99% |
| P2238-HER | 89% | 97% | 98% | 90% | 87% | 91% | 94% | 56% | 75% | 95% | 97% |
| P2262-HER | 90% | 100% | 99% | 90% | 91% | 91% | 97% | 57% | 76% | 97% | 99% |
| P2316_HER | 90% | 100% | 99% | 90% | 91% | 91% | 97% | 57% | 76% | 97% | 99% |
| P2380_HER | 90% | 100% | 99% | 90% | 91% | 91% | 97% | 57% | 76% | 97% | 99% |
| P2753_MUC1 | 90% | 100% | 99% | 90% | 91% | 91% | 97% | 57% | 76% | 97% | 99% |
| P3698_MUC1 | 57% | 93% | 93% | 76% | 77% | 83% | 80% | 23% | 50% | 79% | 80% |
| P3825_MUC1 | 90% | 100% | 99% | 90% | 91% | 91% | 97% | 57% | 76% | 97% | 99% |
| P3776_MUC1 | 57% | 96% | 97% | 83% | 81% | 87% | 87% | 26% | 63% | 93% | 92% |
| P4020_hTERT | 90% | 100% | 99% | 90% | 91% | 91% | 97% | 57% | 76% | 97% | 99% |
| P4121_hTERT | 90% | 100% | 99% | 90% | 91% | 91% | 97% | 57% | 76% | 97% | 99% |
| P4345_hTERT | 89% | 97% | 98% | 90% | 87% | 91% | 94% | 56% | 75% | 95% | 97% |
| P4373_hTERT | 90% | 100% | 99% | 90% | 91% | 91% | 97% | 57% | 76% | 97% | 99% |
| P4453_hTERT | 90% | 100% | 99% | 90% | 91% | 91% | 97% | 57% | 76% | 97% | 99% |
| P4540_hTERT | 90% | 100% | 99% | 90% | 91% | 91% | 97% | 57% | 76% | 97% | 99% |
| P4575_hTERT | 90% | 100% | 99% | 90% | 91% | 91% | 97% | 57% | 76% | 97% | 99% |
| P4616_hTERT | 89% | 97% | 98% | 90% | 87% | 91% | 94% | 56% | 75% | 95% | 97% |
| P4650_hTERT | 89% | 97% | 98% | 90% | 87% | 91% | 94% | 56% | 75% | 95% | 97% |
| P4695_hTERT | 89% | 97% | 98% | 90% | 87% | 91% | 94% | 56% | 75% | 95% | 97% |
| P4759_hTERT | 90% | 100% | 99% | 90% | 91% | 91% | 97% | 57% | 76% | 97% | 99% |
| P4862_TERT | 90% | 100% | 99% | 90% | 91% | 91% | 97% | 57% | 76% | 97% | 99% |
| P4939_hTERT | 57% | 96% | 97% | 83% | 81% | 87% | 87% | 26% | 63% | 93% | 92% |
| P5075_hTERT | 90% | 100% | 99% | 90% | 91% | 91% | 97% | 57% | 76% | 97% | 99% |
| P5400_MAGE1 | 90% | 100% | 99% | 90% | 91% | 91% | 97% | 57% | 76% | 97% | 99% |
| P5232_MAGE1 | 90% | 100% | 99% | 90% | 91% | 91% | 97% | 57% | 76% | 97% | 99% |
| P5525_LAGE1 | 90% | 100% | 99% | 90% | 91% | 91% | 97% | 57% | 76% | 97% | 99% |
| P5566_LAGE1 | 89% | 97% | 98% | 90% | 87% | 91% | 94% | 56% | 75% | 95% | 97% |
| P-HAGE | 90% | 100% | 99% | 90% | 91% | 91% | 97% | 57% | 76% | 97% | 99% |
| P75-P53 | 0% | 0% | 0% | 0% | 0% | 0% | 0% | 0% | 0% | 0% | 0% |
| P750- NYESO | 0% | 0% | 0% | 0% | 0% | 0% | 0% | 0% | 0% | 0% | 0% |
| P1692-HER | 6% | 27% | 31% | 1% | 8% | 6% | 16% | 1% | 6% | 17% | 27% |
| P3150-MUC | 0% | 0% | 0% | 0% | 0% | 0% | 0% | 0% | 0% | 0% | 0% |
| P5449-LAGE | 0% | 0% | 0% | 0% | 0% | 0% | 0% | 0% | 0% | 0% | 0% |

### Example 2

### Peptide-Specific T Cell Responses in Healthy Subjects

Peptide-specific T cell responses were evaluated in peripheral mononuclear cells obtained from healthy subjects. After thawing, PBMC were cultured in 24-well flat bottom plates (or 48-well bottom plates) at a density of 1 million cells per ml in the presence of culture medium (RPMI 1640 Medium, Glutamax™ (Life Technologies) with 5% (v/v) human AB Serum (PAA Laboratories Ltd)) and a pool of peptides (composed of P103-P53, P513_MAGE3, P679_MAGE3, P805_NY_ESO1, P1575_HER, P2238-HER, P3825_MUC1, P3776_MUC1, P4540_hTERT, P4575_hTERT, P5400_MAGE1, P-HAGE) at a total final concentration of 20 μg/mL.

The plates were incubated at 37° C. in a 5% CO₂ incubator and on Day 4, recombinant human IL-2 and IL-15 (R&D Systems) were added to each well at final concentrations of 10 IU/ml and 10 ng/mL respectively. On Day 7, PBMC cultures were removed and washed in culture medium and replaced back into wells in culture medium containing 10 IU/mL of IL-2. After overnight resting at 37° C. in a 5% CO₂ incubator, cells were removed, counted and assessed for viability. PVDF plates (MSIPS4510, Millipore) were coated with anti-human IFN-γ antibody (R&D systems) and incubated overnight at 4° C. Plates were then washed and incubated with blocking buffer (1% BSA (PAA), 5% sucrose (Fisher), Dulbecco's-PBS (Invitrogen)) for at least 1 hour and finally washed with culture medium before use. Cultured PBMCs were dispatched in pre-coated ELISpot plates at a density of 50,000 cells/well in the presence of culture medium (RPMI 1640 Medium, Glutamax™ (Life Technologies) with 5% (v/v) human AB Serum (PAA Laboratories Ltd)) alone (in duplicate), or with pools of overlapping 18-mer peptides representing peptides (P103-P53, P513_MAGE3, P679_MAGE3, P805_NY_ESO1, P1575_HER, P2238-HER, P3825_MUC1, P3776_MUC1, P4540_hTERT, P4575_hTERT, P5400_MAGE1, P-HAGE) at a concentration of 5 μg/peptide/mL or media alone and tested in duplicate (or triplicate). After 18 hours of culture, plates were washed and incubated with a biotinylated secondary anti-human IFN-γ antibody (R&D systems) followed by streptavidin-AP. Production of IFN-γ was detected using the ELISpot blue colour module (R&D Systems) as per manufacturer's instructions. Plates were scanned and wells were counted using an automated ELISpot plate reading system equipped with spot counting software (Cellular Technology Limited). Results are expressed as Spot Forming Cells (SFC)/10⁶ PBMC and background responses represented by IFNγ SFC of PBMC in culture medium alone have been subtracted for each subject. Positive response was defined as number of Spot Forming Cells (SFC)/10⁶ PBMC greater than 20.

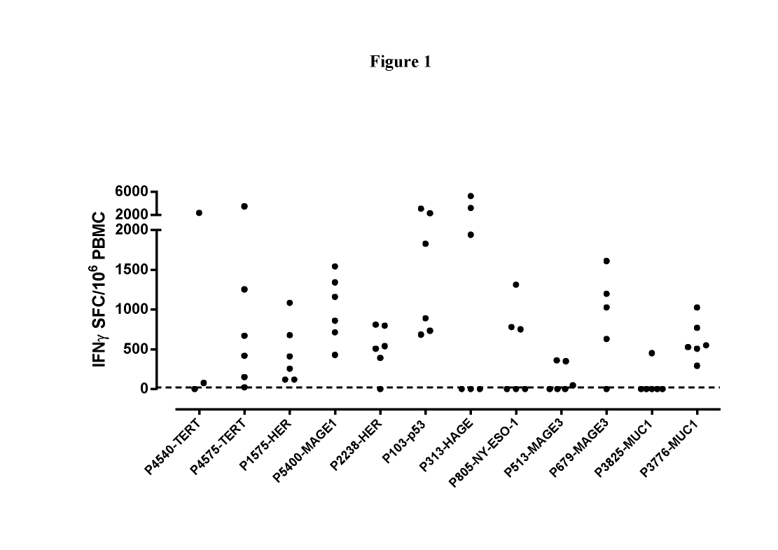

As shown in FIG. 1, the frequency of responding subjects to the specific peptides are 100% for P103-P53, 50% for P513_MAGE3, 80% for P679_MAGE3, 50% for P805_NY_ESO1, 100% for P1575_HER, 83% for P2238-HER, 17% for P3825_MUC1, 100% for P3776_MUC1, 67% for P4540_hTERT, 100% for P4575_hTERT, 100% for P5400_MAGE1, 50% for P-HAGE).

### Example 3

### Peptide-specific T cell responses in Non-Small Cell Lung Cancer (NSCLC) Patients and Healthy Subjects

### Materials and Methods

### Populations

68 subjects clinically diagnosed with Non-Small Cell Lung Cancer (NSCLC) and 40 age-matched healthy individuals were enrolled into a research ethics committee (REC) study at the University Hospital Southampton and The Royal Marsden London. The subject demographics are summarised in the table below:

| | | | Adenocarcinoma/ |
|---|---|---|---|
| | No of | Median age | Squamous-cell |
| Category | subjects | (range) | carcinoma/other |
| Healthy | 40 | 60 (34-84) | — |
| NSCLC Stage I | 2 | 64.5 (56-73) | 2/0/0 |
| NSCLC Stage II | 5 | 66 (45-70) | 4/1/0 |
| NSCLC Stage IIIa | 16 | 65 (37-73) | 10/5/1 |
| NSCLC Stage IIIb | 11 | 63 (54-79) | 3/6/2 |
| NSCLC Stage IV | 34 | 68 (33-78) | 28/6/0 |

Isolation and cryopreservation of PBMC from whole blood

Following written approved consent from all individuals, heparinized, fresh venous blood was collected and Peripheral Blood Mononuclear Cells (PBMCs) were isolated and cryopreserved within 18 hours of blood collection. PBMC were isolated by dilution of blood in an equal volume in Dulbecco's Phosphate Buffered Saline (dPBS, Invitrogen), careful layering onto Lymphoprep (Axis-Shield) and centrifugation at 800×g for 20 min. The PBMC layer was washed in RPMI-1640 medium (Invitrogen) and PBMC were cryopreserved in aliquots of 0.5-1.5×10⁷ cells in 10% DMSO (Sigma Aldrich) in heat-inactivated US-origin Foetal Calf Serum (FCS, A15-204, PAA). Cells were stored in liquid nitrogen until analysis.

Short-term culture of PBMC

Two vials of PBMC from each subject were thawed and lymphocyte numbers were determined using TruCount (BD Biosciences). PBMC were split into two culture conditions (culture 1 and culture 2) and each was stimulated with a mixture of 13 peptide pools. Each peptide pool contains between 3 to 5 peptides having an average peptide length of 18 amino-acids. Each peptide pool corresponds to one of the longer peptides of SEQ ID No 1, 2, 3, 4, 5, 6, 7, 8, 11, 12, 13, 17, 18, 22, 23, 24, 28, 29, 30, 31, 32, 36, 39, 40 and 46. For example, the peptide pool designated “P-P4020-TERT” is the pool corresponding to peptide “P4020-TERT”, that is the peptide of SEQ ID NO: 8. For P-P550-MAGE3, P-P5232-MAGE1, P-P991-SURVIVIN and P-P-HAGE, the N-terminal peptide in the pool has been elongated by one N-terminal amino-acid to facilitate synthesis and improve its solubility. The single amino-acid elongation is not expected to alter the functional properties of these pools relative to their respective peptides.

Culture 1 consisted of pools corresponding to P4020-TERT, P4540-TERT, P4575-TERT, P4616-TERT, P4682-TERT, P513-MAGE3, P550-MAGE3, P679-MAGE3, P991-SURVIVIN, P1331-WT1, P5525-LAGE1.

Culture 2 consisted of pools corresponding to P2753-MUC1, P3698-MUC1, P3776-MUC1, P3825-MUC1, P750-NYESO, P805-NYESO, P830-NYESO, P5232-MAGE1, P5400-MAGE1, P1575-HER, P2238-HER, P103-P53, P-HAGE.

The different peptide pools are summarised in the following table and the individual sequences of the short peptides in each pool are shown in Annex B.

| | | Corresponding | |
|---|---|---|---|
| | | peptide | |
| Culture | Peptide pool | SEQ ID No | Short peptides in pool |
| 1 | P-P4020-TERT | 8 | P4020_hTERT-1, P4020_hTERT-2, |
| | | | P4020_hTERT-3, P4020_hTERT-4, |
| | | | P4020_hTERT-5 |
| 1 | P-P4540-TERT | 17 | P4540-TERT_1, P4540-TERT_2, |
| | | | P4540-TERT_3, P4540-TERT_4, |
| | | | P4540-TERT_5 |
| 1 | P-P4575-TERT | 18 | P4575-TERT_1, P4575-TERT_2, |
| | | | P4575-TERT_3, P4575-TERT_4, |
| | | | P4575-TERT_5 |
| 1 | P-P4616-TERT | 11 | P4616_hTERT-1, P4616_hTERT-2, |
| | | | P4616_hTERT-3, P4616_hTERT-4 |
| 1 | P-P4650-TERT | 12 | P4650_hTERT-1, P4650_hTERT-2, |
| | | | P4650_hTERT-3, P4650_hTERT-4, |
| | | | P4650_hTERT-5 |
| 1 | P-P4682-TERT | 13 | P4862_hTERT-1, P4862_hTERT-2, |
| | | | P4862_hTERT-3, P4862_hTERT-4 |
| 1 | P-P513-MAGE3 | 1 | P513-MAGE-3_1, P513-MAGE-3_2, |
| | | | P513-MAGE-3_3, P513-MAGE-3_4 |
| 1 | P-P550-MAGE3 | 2 | P550_MAGE3-1, P550_MAGE3-2, |
| | | | P550_MAGE3-3, P550_MAGE3-4 |
| 1 | P-P590-MAGE3 | 48 | P590_MAGE3-1, P590_MAGE3-2, |
| | | | P590_MAGE3-3 |
| 1 | P-P679-MAGE3 | 3 | P679-MAGE-3_1, P679-MAGE-3_2, |
| | | | P679-MAGE-3_3, P679-MAGE-3_4 |
| 1 | P-P991-SURVIVIN | 30 | P991_SURVIVIN-1, P991_SURVIVIN-2, |
| | | | P991_SURVIVIN-3, P991_SURVIVIN-4 |
| 1 | P-P1331-WT1 | 31 | P1331_WT1-1, P1331_WT1-2, |
| | | | P1331_WT1-3, P1331_WT1-4 |
| 1 | P-P5525-LAGE1 | 39 | P5525_LAGE1-1, P5525_LAGE1-2, |
| | | | P5525_LAGE1-3, P5525_LAGE1-4 |
| 2 | P-P2753-MUC1 | 4 | P2753_MUC1-1, P2753_MUC1-2, |
| | | | P2753_MUC1-3 |
| 2 | P-P3698-MUC1 | 7 | P3698_MUC1-1, P3698_MUC1-2, |
| | | | P3698_MUC1-3, P3698_MUC1-4, |
| | | | P3698_MUC1-5 |
| 2 | P-P3776-MUC1 | 6 | P3776-MUC-1_1, P3776-MUC-1_2, |
| | | | P3776-MUC-1_3, P3776-MUC-1_4 |
| 2 | P-P3825-MUC1 | 5 | P3825-MUC-1_1, P3825-MUC-1_2, |
| | | | P3825-MUC-1_3, P3825-MUC-1_4, |
| | | | P3825-MUC-1_5 |
| 2 | P-P750-NYESO | 46 | P750-NYESO-1, P750-NYESO-2, |
| | | | P750-NYESO-3, P750-NYESO-4 |
| 2 | P-P805-NYESO | 28 | P805-NY-ESO-1_1, P805-NY-ESO-1_2, |
| | | | P805-NY-ESO-1_3, P805-NY-ESO-1_4 |
| 2 | P-P830-NYESO | 29 | P830_NY_ESO1-1, P830_NY_ESO1-2, |
| | | | P830_NY_ESO1-3, P830_NY_ESO1-4 |
| 2 | P-P5232-MAGE1 | 23 | P5232_MAGE1-1, P5232_MAGE1-2, |
| | | | P5232_MAGE1-3, P5232_MAGE1-4 |
| 2 | P-P5400-MAGE1 | 22 | P5400-MAGE1_1, P5400-MAGE1_2, |
| | | | P5400-MAGE1_3, P5400-MAGE1_4, |
| | | | P5400-MAGE1_5 |
| 2 | P-P1575-HER | 32 | P1575-HER_1, P1575-HER_2, |
| | | | P1575-HER_3, P1575-HER_4 |
| 2 | P-P2238-HER | 36 | P2238-HER_1, P2238-HER_2, |
| | | | P2238-HER_3, P2238-HER_4, |
| | | | P2238-HER_5 |
| 2 | P-P103-P53 | 24 | P103-P53_1, P103-P53_2, |
| | | | P103-P53_3, P103-P53_4 |
| 2 | P-P-HAGE | 40 | P313-HAGE_1, P313-HAGE_2, |
| | | | P313-HAGE_3, P313-HAGE_4 |

PBMC were cultured in 2 mL culture medium (CM, RPMI1640 Glutamax supplemented with 5% untreated human AB serum (PAA) and gentamicin) in 24 well cell culture plates at a concentration of 1×10⁶ cells/mL for a total of 7 days. Each pool was added at 20 μg/mL total peptide final concentration. On day 1, IL-7 (Peprotech) and IL-2 (R&D Systems) were added to the cultures to a final concentration of 5 ng/mL and 10 IU/mL respectively. On day 3, IL-2 was added at a concentration of 10 IU/mL. On day 7, cells were harvested from cell culture plates washed with culture medium and counted using TruCount (BD Biosciences) before analysis using IFNγ (interferon gamma) ELISpot assay or intracellular cytokine staining (ICS) as described below.

Note, P-P750-NYESO was included as a negative control peptide.

### Human IFNγ ELISpot Assay

96 well multiscreen PVDF filter plates (Millipore) were coated overnight at 2-8° C. with 100 μL (1:80) of anti-human IFNγ capture mAb (SEL285, R&D Systems). Plates were then washed and blocked with PBS supplemented with 1% BSA (bovine serum albumen, PAA) and 5% sucrose for between 2 hr and 7 days at 2-8° C. Cells from the short-term cultures were plated in duplicate (assay controls were tested in triplicate) wells at 0.5-1×10⁵ cells per well. 26 peptide pools were tested individually, namely P—P-HAGE, P-P4650_hTERT, P-P4020_hTERT, P-P5525_LAGE1, P-P4540_hTERT, P-P830_NY_ESO1, P-P805_NY_ESO1, P-P679_MAGE3, P-P5400_MAGE1, P-P550_MAGE3, P-P103_P53, P5232_MAGE1, P-P4575_hTERT, P-P4616_hTERT, P513_MAGE3, P-P1575_HER, P-P590_MAGE3, P-P991_SURVIVIN, P-P3698_MUC1, P-P2238-HER, P-P3825_MUC1, P-P4862_hTERT, P-P1331_WT1, P-P2753_MUC1, P-P3776_MUC1 and P-P750-NYESO as a negative control peptide. Final antigen concentrations used were: for the 26 peptide pools, individual peptides at a concentration of 5 μg/peptide/mL; for the PHA positive control, 2.5 μg/mL. The assay negative control was cells plates with culture medium alone (no antigen). ELISpot plates were incubated for 18-18.5 hours at 37° C./5% CO₂. Plates were then washed and incubated with 100 μL (1:80) detection mAb (SEL285, R&D Systems) for 2 hrs at room temperature (RT). Following washing plates were incubated with a streptavidin-conjugated alkaline phosphatase (1:80) for 1 hr followed by BCIP/NBT substrate for 30 min according to the manufacturer's instructions (SEL002, R&D Systems). ELISpot plates were scanned and counted using an automated plate counting system (CTL ImmunoSpot).

### Intracellular Cytokine Staining (ICS)

Short term culture of PBMC were prepared as described above, except culture conditions 1 and 2 were replaced with alternative culture conditions 1 to 5 below, each of which involves stimulation with a different combination of peptide pools:

Culture 1: P-P5525-LAGE-1, P-P103-P53, P-P830-NYESO-01, P-P-HAGE, P-P5232-MAGE-A1, P-P550-MAGE-A3 and P-P4616-hTERT;

Culture 2: P-P5525-LAGE-1, P-P103-P53, P-P830-NYESO-01, P-P-HAGE, P-P5232-MAGE-A1, P-P550-MAGE-A3, P-P4616-hTERT, P-P805_NY_ESO1, P-P-P679_MAGE3, P-P4650_hTERT, P-P4575_hTERT, and P-P4020_hTERT;

Culture 3: P-P805_NY_ESO1, P-P5400_MAGE1, P-P679_MAGE3, P-P4650_hTERT, P-P4575_hTERT, P-P4540_hTERT, P-P550_MAGE3 and P-P4020_hTERT;

Culture 4: P-P550_MAGE3;

Culture 5: P-P805_NY_ESO1

Cells from the short-term cultures were plated in a 96 well round bottom plate at 5×10⁵ PBMC/well stimulated with individual peptide pools used at a concentration of 5 μg/peptide/mL. Plates were incubated at 37° C. in a 5% CO2 incubator for 20h. PMA/Ionomycin was added to respective wells and Golgi plug (BD Biosciences) was added to all wells after the first 3h of the assay. The cells were harvested and washed with PBS+0.1% BSA (wash buffer) and stained with anti-CD3, anti-CD4 and anti-CD8 (BD Biosciences) for 30 minutes at 4° C. After another wash, the cells were fixed and permeabilised with 100 μL of Cytofix/Cytoperm solution (BD Biosciences) for 20 minutes at 4° C., followed by two washes with 1× Perm/Wash solution (BD Biosciences). Finally, cells were stained with ant-IL-2-FITC, anti-IFNγ-PE and anti-TNFα PerCP-Cy5.5 (BD Biosciences) for 30 minutes at 4° C. Samples were acquired on a FACSCanto II flow cytometer (BD Biosciences). Gating was based on isotype control antibody staining and media stimulated samples for each subject.

### Results and Discussion

25 peptide sequences (P-HAGE, P4650_hTERT, P4020_hTERT, P5525_LAGE1, P4540_hTERT, P830_NY_ESO1, P805_NY_ESO1, P679_MAGE3, P5400_MAGE1, P550_MAGE3, P103_P53, P5232_MAGE1, P4575_hTERT, P4616_hTERT, P513_MAGE3, P1575 HER, P590-MAGE3, P991 SURVIVIN, P3698 MUC1, P2238-HER, P3825 MUC1, P4862_hTERT, P1331 WT1, P2753 MUC1, P3776 MUC1) falling under the definition of preferred peptides based the analysis of population coverage as presented in example 1 were selected to be tested for their ability to stimulate T cells from Healthy volunteers and NSCLC patients. One peptide sequence (P750-NYESO) which did not fall under the definition of preferred peptides from example 1 was used as a negative control peptide.

The T cell assay methodology is based on the use of PBMC obtained using the same isolation and preservation for all samples. In addition, aged-matched samples from NSCLC patients and healthy subjects were used to allow for an appropriate comparison between the two groups. Healthy subjects and NSCLC patients were mainly of Caucasian ethnicity (˜85%) with a smaller proportion of subjects with from ethnicities including Asian, Black, and Oriental. As a result of unselected recruitments of NSCLC patients and Healthy subjects, it is anticipated that the in T cell responses observed across the PBMC samples are not biased towards any specific HLA class I and HLA class II molecules but are either representative of the HLA polymorphism in the general population.

Due to the very low frequency of T cell specific for tumour antigens in the peripheral blood of healthy and cancer patients (data not shown), a methodology based on a short term 7 days in vitro culture has been used as described in the materials and methods. The short term culture results in the expansion of antigen-specific T cell precursors. The frequency of antigen-specific T cells was measured by mean of an IFN-g ELIspot assay as described in the materials and methods. The frequency of antigen-specific T cells was measured using pools of overlapping short peptides (average peptide length 18 amino-acids, 3 to 5 peptides per pool), each corresponding to a longer peptide of interest. The immune responses measured with the pool of peptides is considered to be representative of the corresponding long peptides of interest. A stringent threshold of 500 spots per million PBMCs has been established to identify positive peptide responses.

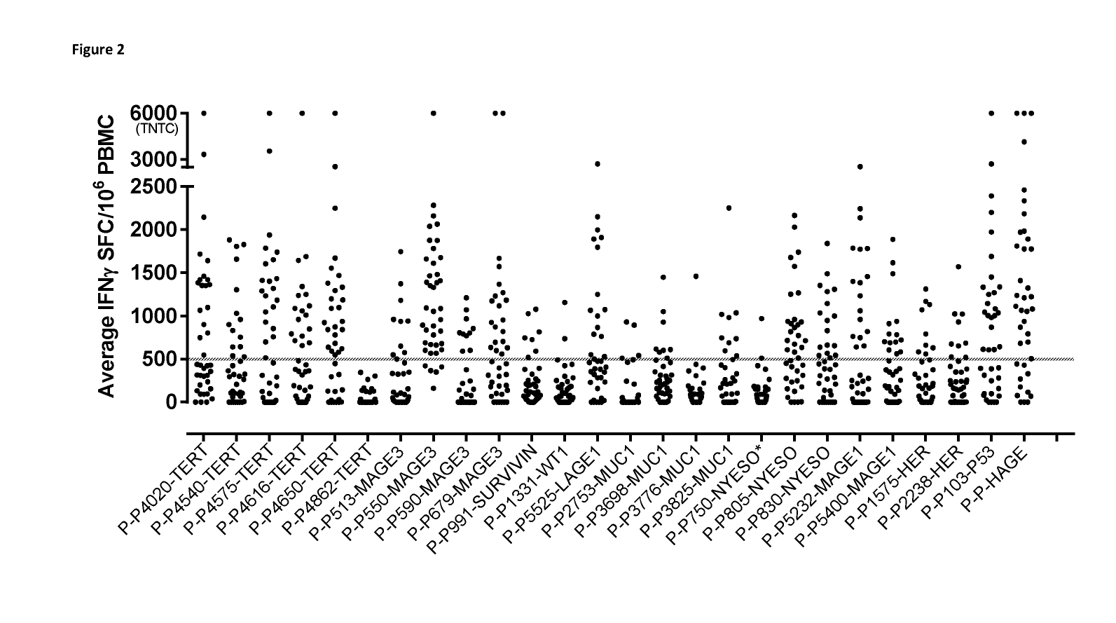

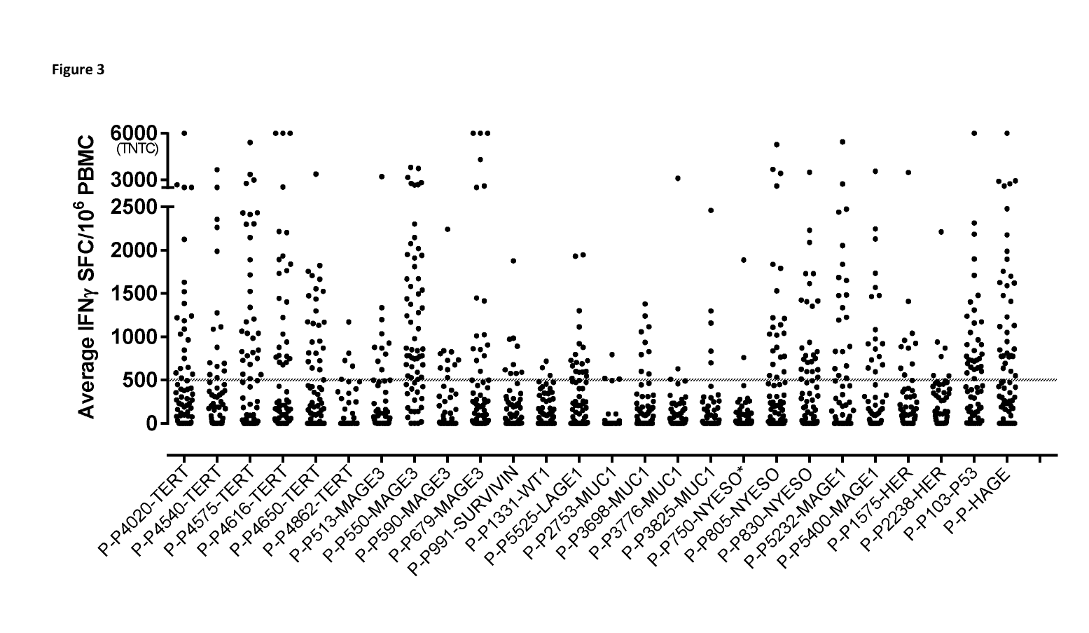

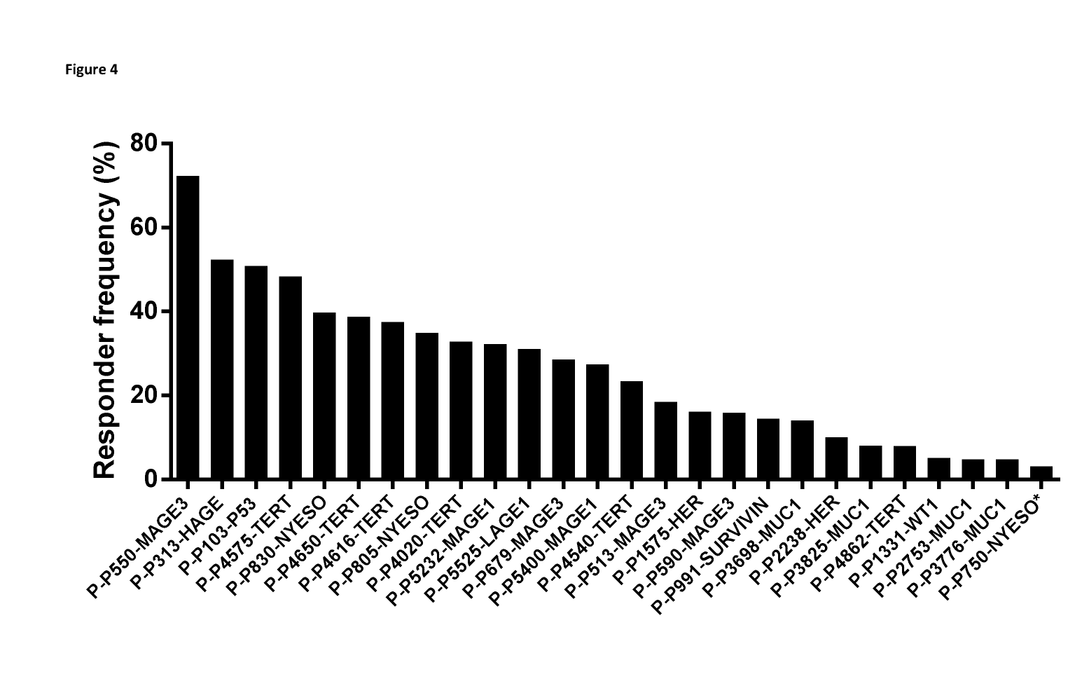

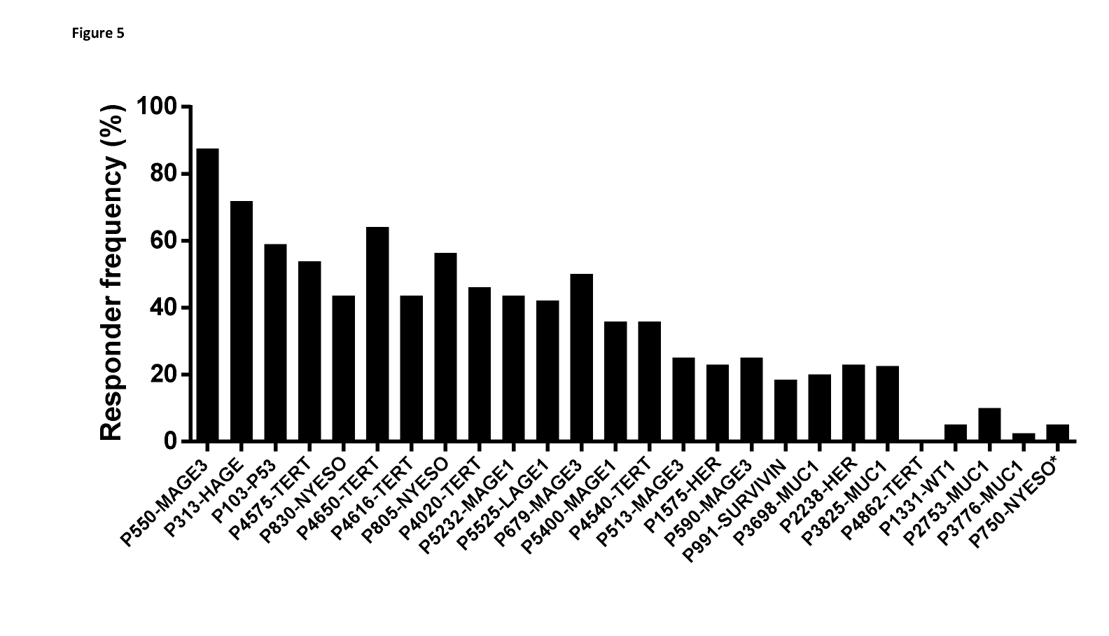

The 25 peptides vary with regard to their respective magnitude of response (FIG. 2 and FIG. 3) and responder frequency (FIG. 4 and FIG. 5) across the two groups tested. Interestingly, there is no obvious correlation between the observed responder frequency in NSCLC patients and healthy subjects and the predicted population coverage across any of the different ethnic groups presented in Example 1. Nevertheless, the negative control peptide P750-NYESO having a low predicted population coverage across all ethnic groups (as shown in Example 1) was demonstrated to be amongst the lowest responding peptides in term of magnitude and responder frequency (FIGS. 2, 3, 4 and 5).

Three groups of peptides have been defined based on their responder frequency observed in NSCLC patients as shown in FIG. 3. GROUP 1: High responding peptides: responder frequency >20%: P-HAGE, P4650_hTERT, P4020_hTERT, P5525_LAGE1, P4540_hTERT, P830_NY_ESO1, P805_NY_ESO1, P679_MAGE3, P5400_MAGE1, P550_MAGE3, P103_P53, P5232_MAGE1, P4575_hTERT, P4616_hTERT; GROUP 2: Moderate responding peptides: responder frequency between 10 and 20%: P513_MAGE3, P1575_HER, P590-MAGE3, P991_SURVIVIN, P3698_MUC1, P2238-HER; GROUP 3: Low responding peptides—responder frequency <10%: P3825_MUC1, P3776_MUC1, P2753_MUC1, P4862_hTERT, P1331_WT1, P750-NYESO. Tables A1, A2 and A3 below summarise the three groups and provide the corresponding SEQ ID NOs.

TABLE A1
GROUP 1: High responding peptides: responder frequency >20%:

| Name | SEQ ID NO | Frequency |
|---|---|---|
| P-HAGE | 40 | 52.38% |
| P4020_hTERT | 8 | 32.81% |
| P5525_LAGE1 | 39 | 31.03% |
| P4540_hTERT | 17 | 23.43% |
| P830_NY_ESO1 | 29 | 39.68% |
| P4650_hTERT | 12 | 38.70% |
| P805_NY_ESO1 | 28 | 34.92% |
| P679_MAGE3 | 3 | 28.57% |
| P5400_MAGE1 | 22 | 27.41% |
| P550_MAGE3 | 2 | 72.30% |
| P103_P53 | 24 | 50.81% |
| P5232_MAGE1 | 23 | 32.25% |
| P4575_hTERT | 18 | 48.38% |
| P4616_hTERT | 11 | 37.50% |

TABLE A2
GROUP 2: Moderate responding peptides:
responder frequency between 10 and 20%:

| Name | SEQ ID NO | Frequency |
|---|---|---|
| P513_MAGE3 | 1 | 18.46% |
| P590-MAGE3 | 48 | 15.87% |
| P991_SURVIVIN | 30 | 14.51% |
| P2238-HER | 36 | 10.00% |
| P1575_HER | 32 | 16.12% |
| P3698_MUC1 | 7 | 14.06% |

TABLE A3
GROUP 3: Low responding peptides - responder frequency <10%:

| Name | SEQ ID NO | Frequency |
|---|---|---|
| P3825_MUC1 | 5 | 8.06% |
| P1331_WT1 | 31 | 5.08% |
| P2753_MUC1 | 4 | 4.76% |
| P4862_hTERT | 13 | 7.93% |
| P3776_MUC1 | 6 | 4.76% |
| P750-NYESO | 46 | 3.12% |

Peptides in group 1 are preferred for inclusion in a composition for the treatment or prevention of cancer, alone or in combination. Peptides in group 2 are moderately preferred. Peptides in group 3 are less preferred peptides, since they have a level of response close to the negative control peptide P750-NYESO.

As is shown, some target tumour antigens are more frequently recognized than others. For example Telomerase contains 5 out of 6 peptides in group 1 as opposed to MUC-1 containing one peptide in group 2 and three peptides in group 3. Factors influencing this phenomenon may be related to the level of expression of the antigen in normal and tumour cells, the cellular compartment in which the protein is expressed, immune tolerance, T cell exhaustion, or T cell regulatory mechanisms exerted against the antigen.

These factors may vary from one individual to another and during the course of the disease. Interestingly, different peptides derived from the same tumour antigen may present different degree of response. For example, P4650_hTERT, P4020_hTERT, P4540_hTERT, P4575_hTERT, P4616_hTER from telomerase belong to group 1 as opposed to P4862_hTERT in group 3. Similarly, P679_MAGE3, P550_MAGE3 from MAGE 3 belong to group 1 as opposed to P590-MAGE3, P513_MAGE3 in group 2. As expected, the negative control peptide P750-NYESO derived from NY-ESO-1 is placed in group 3, by contrast to P830_NY_ESO1 and P805_NY_ESO1 which are placed in group 1.

Differences between the immune responses in NSCLC patients and age-matched healthy individuals were observed. Globally, the frequency of antigen-specific T cells for a majority of peptides is lower in NSCLC patient compared to Healthy subjects (FIG. 5). This higher level of immune response observed in healthy subjects may reflect the role of tumour antigen-specific T cell in the immune surveillance against cancer, a defence mechanism which presumably is altered in NSCLC patients. A similar phenomenon is thus anticipated in other cancer indications in which the selected tumour antigens (MAGE-3, MAGE-1, Telomerase, HAGE, LAGE, HER-2/neu, MUC-1, P53, NY-ESO-1, LAGE-1, Survivin) are also expressed. Such cancer indications include breast cancer, hepatic cancer, brain cancer, stomach cancer, pancreatic cancer, kidney cancer, ovarian cancer, myeloma, acute myelogenous leukaemia, chronic myelogenous leukaemia, head and neck cancer, colorectal cancer, renal cancer, oesophageal cancer, melanoma skin cancer and prostate cancer,

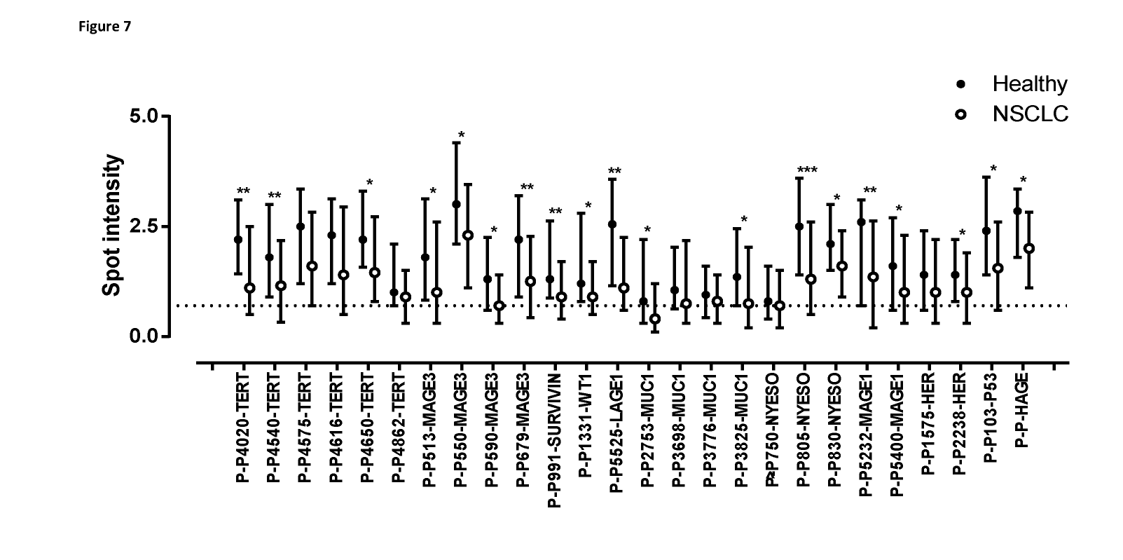

Surprisingly, the intensity of the spots measured in the IFN-γ ELIspot assay appear to be significantly reduced for a number of peptides tested in NSCLC patients compared with healthy subjects (see FIG. 7), reflecting a tumour antigen-specific T cell defect as a result of the disease. The spot intensity reflects the quantity of IFN-g production at a single cell level. A majority of peptides from group 1 (P-HAGE (SEQ ID NO:40), P4020_hTERT (SEQ ID NO:8), P5525_LAGE1 (SEQ ID NO:39), P4540_hTERT (SEQ ID NO:17), P830_NY_ESO1 (SEQ ID NO:29), P4650_hTERT (SEQ ID NO:12), P805_NY_ESO1 (SEQ ID NO:28), P679_MAGE3 (SEQ ID NO:3), P5400_MAGE1 (SEQ ID NO:22), P550_MAGE3 (SEQ ID NO:2), P103_P53 (SEQ ID NO:24), P5232_MAGE1 (SEQ ID NO:23)) present spot intensities significantly lower in NSCLC patients compared with Healthy subjects. These group 1 peptides are particularly preferred for inclusion in a composition for the treatment of cancer. Similar results were obtained with P513_MAGE3, P590-MAGE3, P991_SURVIVIN, P2238-HER from group 2, which are thus the most preferred peptides from that group.

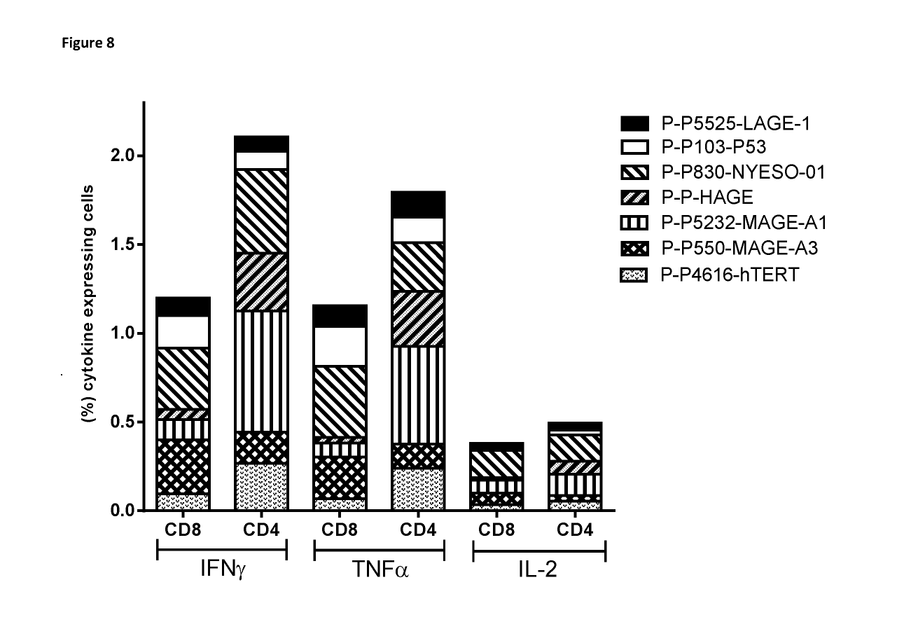

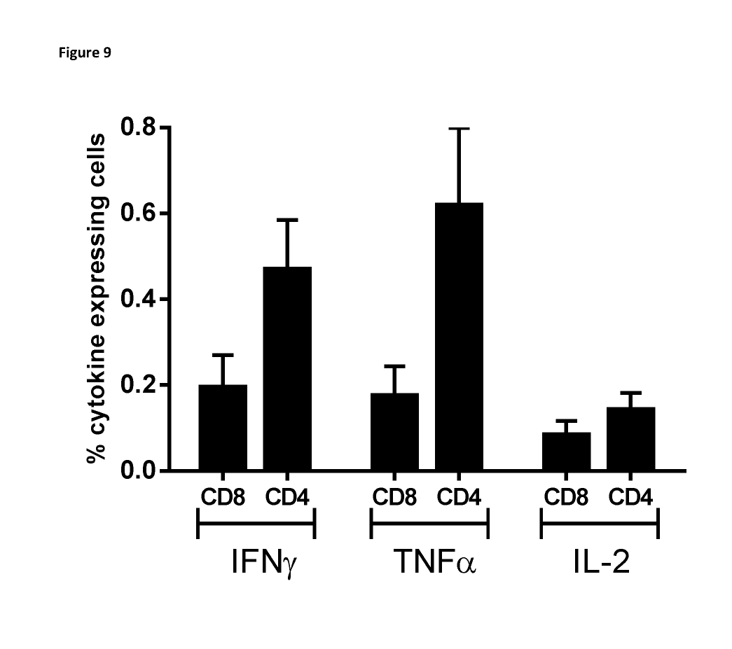

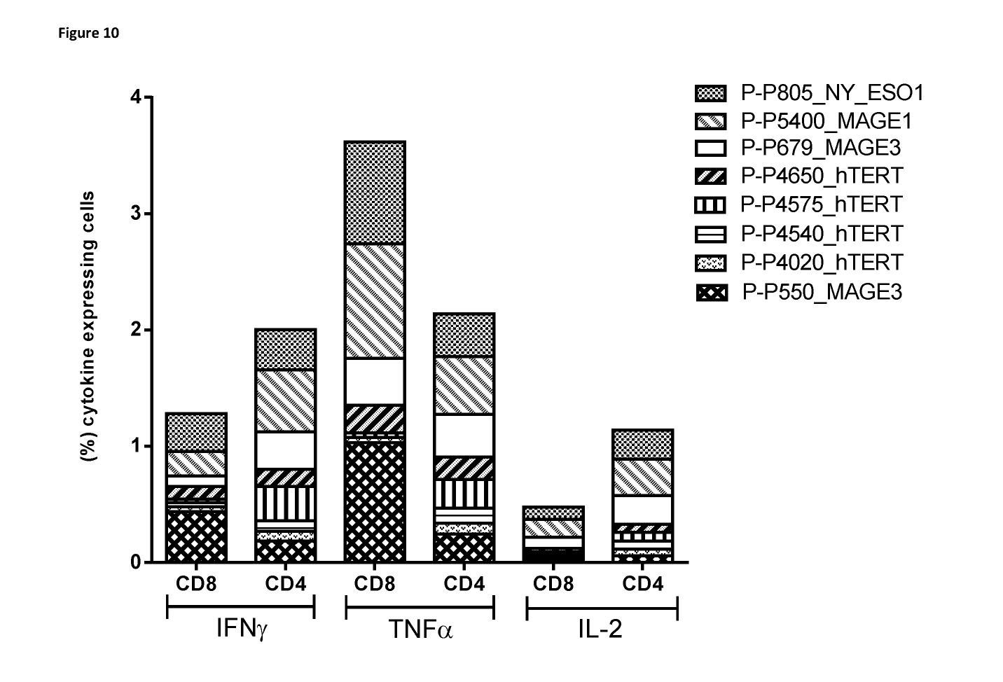

In addition, peptide P-HAGE, P4650_hTERT, P4020_hTERT, P5525 LAGE1, P4540_hTERT, P830_NY_ESO1, P805 NY_ESO1, P679_MAGE3, P5400_MAGE1, P550_MAGE3, P103_P53, P5232_MAGE1, P4575_hTERT and P4616_hTERT show the ability to promote Th1 cytokine-producing CD4 and/or CD8 T cell responses as measured by intracellular cytokine staining (see FIGS. 8, 9 and 10).

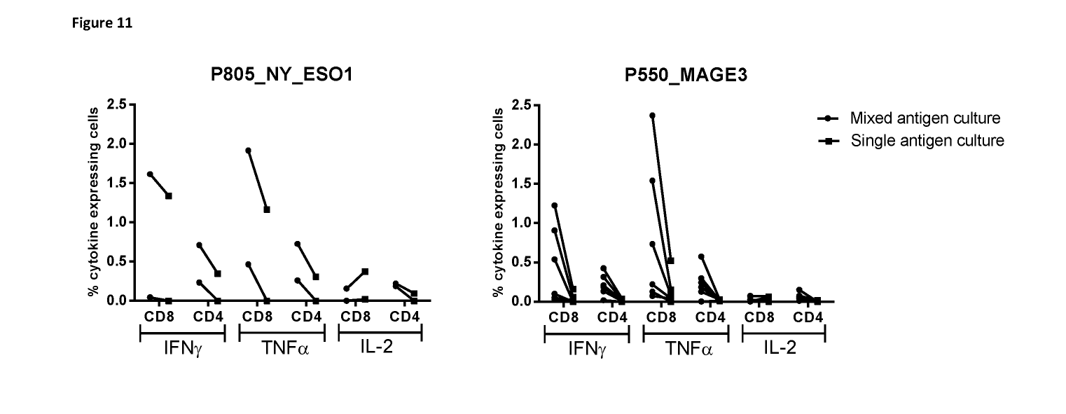

Surprisingly, FIG. 11 shows that the magnitude of peptide-specific CD4 and CD8 T cell responses is dramatically lower after short term culture with a single peptide pool (culture 4 with P-P550_MAGE3 only and culture 5 with P-P805_NY_ESO1 only) as opposed to short term culture with a combination of several peptide pools. This suggests that by combining peptides, particularly peptides from tier 1, it is possible to achieve a synergistic improvement in the observed response. This result provides evidence of the absence of competition between the peptides in the mixture, at least during the short term culture phase, and also supports the presence of a “helper” effect of at least some of the peptides in the mixture (particular those promoting the highest level of response: P805_NY_ESO1, P5400_MAGE1 and P550_MAGE3). This effect likely acts through the production of cytokines, chemokines and co-stimulatory factors.

Annex A

| | | |
|---|---|---|
| SEQ ID No 1 | P513_MAGE3 | EFQAALSRKVAELVHFLLLKYRAREPVTKAEMLG |
| SEQ ID No 2 | P550_MAGE3 | SVVGNWQYFFPVIFSKASSSLQLVEGIELMEVDPIGHLYIF |
| SEQ ID No 3 | P679_MAGE3 | YEFLWGPRALVETSYVKVLHHMVKISGGPHISYPPLH |
| SEQ ID No 4 | P2753_MUC1 | MTPGTQSPFFLLLLLTVLTVVTGSGHASSTPGG |
| SEQ ID No 5 | P3825_MUC1 | EMFLQIYKQGGFLGLSNIKFRPGSVVVQLTLAFREGTINVH |
| SEQ ID No 6 | P3776_MUC1 | TSSNHSTSPQLSTGVSFFFLSFHISNLQFNSSLEDPST |
| SEQ ID No 7 | P3698_MUC1 | PALGSTAPPVHNVTSASGSASGSASTLVHNGTSARATTTPASK |
| SEQ ID No 8 | P4020_hTERT | RAVRSLLRSHYREVLPLATFVRRLGPQGWRLVQRGDPAAFRA |
| SEQ ID No 9 | P4121_hTERT | AFTTSVRSYLPNTVTDALRGSGAWGLLLRRVGDDVL |
| SEQ ID No 10 | P4345_hTERT | KEQLRPSFLLSSLRPSLTGARRLVETIFLGSRPWMPGTPRRLPR |
| SEQ ID No 11 | P4616_hTERT | HREARPALLTSRLRFIPKPDGLRPIVNMDYVVGARTFRREK |
| SEQ ID No 12 | P4650_hTERT | RTFRREKRAERLTSRVKALFSVLNYERARRPGLLGASVLGLDDI |
| SEQ ID No 13 | P4862_hTERT | AGIRRDGLLLRLVDDFLLVTPHLTHAKTFLRTLVRGVPEYG |
| SEQ ID No 14 | P5075_hTERT | AVQWLCHQAFLLKLTRHRVTYVPLLGSLRTAQTQLSRK |
| SEQ ID No 15 | P4373_hTERT | LGSRPWMPGTPRRLPRLPQRYWQMRPLFLELLGNHAQCPYG |
| SEQ ID No 16 | P4453_hTERT | RRLVQLLRQHSSPWQVYGFVRACLRRLVPPGLWGSRH |
| SEQ ID No 17 | P4540_hTERT | HRLREEILAKFLHWLMSVYVVELLRSFFYVTETTFQKNR |
| SEQ ID No 18 | P4575_hTERT | TFQKNRLFFYRKSVWSKLQSIGIRQHLKRVQLRELSEAEVR |
| SEQ ID No 19 | P4695_hTERT | RAWRTFVLRVRAQDPPPELYFVKVDVTGAYDTIPQDRLTEVIASII |
| SEQ ID No 20 | P4759_hTERT | HGHVRKAFKSHVSTLTDLQPYMRQFVAHLQETSPLRDAVVI |
| SEQ ID No 21 | P4939_hTERT | GLLLDTRTLEVQSDYSSYARTSIRASLTFNRGFKAGRNMRRK |
| SEQ ID No 22 | P5400_MAGE1 | PARYEFLWGPRALAETSYVKVLEYVIKVSARVRFFFPSLREA |
| SEQ ID No 23 | P5232_MAGE1 | ILESLFRAVITKKVADLVGFLLLKYRAREPVTKAEMLESVIK |
| SEQ ID No 24 | P103_P53 | APSWPLSSSVPSQKTYQGSYGFRLGFLHSGTAKSVTCT |
| SEQ ID No 25 | P154_P53 | PVQLWVDSTPPPGTRVRAMAIYKQSQHMTEVVRR |
| SEQ ID No 26 | P205_P53 | SDGLAPPQHLIRVEGNLRVEYLDDRNTFRHSVVVPYEPPEVGS |
| SEQ ID No 27 | P262_P53 | MGGMNRRPILTIITLEDSSGNLLGRNSFEVRV |
| SEQ ID No 28 | P805_NY_ESO1 | RGPESRLLEFYLAMPFATPMEAELARRSLAQDAPPLPVPG |
| SEQ ID No 29 | P830_NY_ESO1 | LARRSLAQDAPPLPVPGVLLKEFTVSGNILTIRLTAADHRQ |
| SEQ ID No 30 | P991_SURVIVIN | AFLSVKKQFEELTLGEFLKLDRERAKNKIAKETNNKKKE |
| SEQ ID No 31 | P1331_WT1 | GAQYRIHTHGVFRGIQDVRRVPGVAPTLVRSASETSEKRPF |
| SEQ ID No 32 | P1575_HER | VQGYVLIAHNQVRQVPLQRLRIVRGTQLFEDNYALAVL |
| SEQ ID No 33 | P1632_HER | GGLRELQLRSLTEILKGGVLIQRNPQLCYQDTILWKDIFHKNN |
| SEQ ID No 34 | P1930_HER | GRILHNGAYSLTLQGLGISWLGLRSLRELGSGLALIHHNTHL |
| SEQ ID No 35 | P2200_HER | SGAMPNQAQMRILKETELRKVKVLGSGAFGTVYKGIWIPDGEN |
| SEQ ID No 36 | P2238_HER | GENVKIPVAIKVLRENTSPKANKEILDEAYVMAGVGSPYVSRKK |
| SEQ ID No 37 | P2262_HER | KEILDEAYVMAGVGSPYVSRLLGICLTSTVQLVTQLMPYG |
| SEQ ID No 38 | P2316_HER | SQDLLNWCMQIAKGMSYLEDVRLVHRDLAARNVLVKSPNHVKI |
| SEQ ID No 39 | P5525_LAGE1 | RLLQLHITMPFSSPMEAELVRRILSRDAAPLPRPGAVL |
| SEQ ID No 40 | P-HAGE | QTGTGKTLCYLMPGFIHLVLQPSLKGQRNRPGMLV |
| SEQ ID No 41 | p5449-LAGE | AEGQGTGGSTGDADGPGGPGIPDGPGGNAGGPGEAGAT |
| SEQ ID No 42 | P5566_LAGE1 | RPGAVLKDFTVSGNLLFMSVRDQDREGAGRMRVVGWGLGSASP |
| SEQ ID No 43 | p3150-MUC | HGVTSAPDTRPAPGSTAPPAHGVTSAPDTRPAPGSTA |
| SEQ ID No 44 | p1692_HER | CSPMCKGSRCWGESSEDCQSLTRTVCAGGCARCKGPLP |
| SEQ ID No 45 | P2380_HER | KVPIKWMALESILRRRFTHQSDVWSYGVTVWELMTFGAKPY |
| SEQ ID No 46 | P750-NYESO | PDGPGGNAGGPGEAGATGGRGPRGAGAARASGPGGGAP |
| SEQ ID No 47 | P75-P53 | TEDPGPDEAPRMPEAAPPVAPAPAAPTPAAPAPAPSWP |
| SEQ ID No 48 | P590_MAGE3 | LGLSYDGLLGDNQIMPKAGLLIIVLAIIAREGD |

Annex B

| | | | | | |
|---|---|---|---|---|---|
| P-P4020-TERT | | | | | |
| P4020_hTERT-1 | 49 | P4020_hTERT-2 | 50 | P4020_hTERT-3 | 51 |
| RAVRSLLRSHYREVLPLA | | RSHYREVLPLATFVRR | | REVLPLATFVRRLGPQGWRL | |
| P4020_hTERT-4 | 52 | P4020_hTERT-5 | 53 | | |
| TFVRRLGPQGWRLVQRGDPAA | | PQGWRLVQRGDPAAFRA | | | |
| P-P4540-TERT | | | | | |
| P4540-TERT_1 | 54 | P4540-TERT_2 | 55 | P4540-TERT_3 | 56 |
| HRLREEILAKFLHWLMSV | | ILAKFLHWLMSVYVVELLKKK | | HWLMSVYVVELLRSFFYVKKK | |
| P4540-TERT_4 | 57 | P4540-TERT_5 | 58 | | |
| YVVELLRSFFYVTETTFQ | | LRSFFYVTETTFQKNR | | | |
| P-P4575-TERT | | | | | |
| P4575-TERT_1 | 59 | P4575-TERT_2 | 60 | P4575-TERT_3 | 61 |
| TFQKNRLFFYRKSVWSK | | RLFFYRKSVWSKLQSIGI | | KSVWSKLQSIGIRQHLKR | |
| P4575-TERT_4 | 62 | P4575-TERT_5 | 63 | | |
| LQSIGIRQHLKRVQLREL | | RQHLKRVQLRELSEAEVR | | | |
| P-P4616-TERT | | | | | |
| P4616_hTERT-1 | 64 | P4616_hTERT-2 | 65 | P4616_hTERT-3 | 66 |
| HREARPALLTSRLRFIPK | | ALLTSRLRFIPKPDGLRPI | | FIPKPDGLRPIVNMDYVV | |
| P4616_hTERT-4 | | | | | |
| LRPIVNMDYVVGARTFRREK | | | | | |
| P-P4650-TERT | | | | | |
| P4650_hTERT-1 | 68 | P4650_hTERT-2 | 69 | P4650_hTERT-3 | 70 |
| RTFRREKRAERLTSRVKAL | | AERLTSRVKALFSVLNY | | SRVKALFSVLNYERARRPGL | |
| P4650_hTERT-4 | 71 | P4650_hTERT-5 | 72 | | |
| SVLNYERARRPGLLGASV | | ARRPGLLGASVLGLDDI | | | |
| P-P4862-TERT | | | | | |
| P4862_hTERT-1 | 73 | P4862_hTERT-2 | 74 | P4862_hTERT-3 | 75 |
| AGIRRDGLLLRLVDDFLLVT | | LLLRLVDDFLLVTPHLTHA | | FLLVTPHLTHAKTFLRTL | |
| P4862_hTERT-4 | 76 | | | | |
| HLTHAKTFLRTLVRGVPEYG | | | | | |
| P-P513-MAGE3 | | | | | |
| P513-MAGE-3_1 | 77 | P513-MAGE-3_2 | 78 | P513-MAGE-3_3 | 79 |
| EFQALLSRKVAELVHFLL | | SRKVAELVHFLLLKYRAR | | LVHFLLLKYRAREPVTKA | |
| P513-MAGE-3_4 | 80 | | | | |
| LKYRAREPVTKAEMLG | | | | | |
| P-P550-MAGE3 | | | | | |
| P550_MAGE3-1 | 81 | P559_MAGE3-2 | 82 | P550_MAGE3-3 | 83 |
| GSVVGNWQYFFPVIFSK | | WQYFFPVIFSKASSSLQLV | | FSKASSSLQLVFGIELMEV | |
| P550_MAGE3-4 | 84 | | | | |
| LQLVFGIELMEVDPIGHLYIF | | | | | |
| P-P590-MAGE3 | | | | | |
| P590_MAGE3-1 | 85 | P590_MAGE3-2 | 86 | P590_MAGE3-3 | 87 |
| KKKSYDGLLGDNQIMPKAGLLIKKK | | KKKGDNQIMPKAGLLIIVLAIKKK | | KKKAGLLIIVLAIIAREGDKKK | |
| P-P679-MAGE3 | | | | | |
| P679-MAGE-3_1 | 88 | P679-MAGE-3_2 | 89 | P679-MAGE-3_3 | 90 |
| YEFLWGPRALVESTYVKV | | PRALVETSYVKVLHHMVK | | TSYVKVLHHMVKISGGPH | |
| P679-MAGE-3_4 | 91 | | | | |
| LHHMVKISGGPHISYPPLH | | | | | |
| P-P991-SURVIVIN | | | | | |
| P991_SURVIVIN-1 | 92 | P991_SURVIVIN-2 | 93 | P991_SURVIVIN-3 | 94 |
| SAFLSVKKQFEELTLGEFL | | KQFEELTLGEFLKLDRERAKN | | FLKLDRERAKNKIAKETN | |
| P991_SURVIVIN-4 | 95 | | | | |
| RERAKNKIAKETNNKKKE | | | | | |
| P-P1331-WT1 | | | | | |
| P1331_WT1-1 | 96 | P1331_WT1-2 | 97 | P1331_WT1-3 | 98 |
| GAQYRIHTHGVFRGIQDVR | | HGVFRGIQDVRRVPGV | | GIQDVRRVPGVAPTLVRS | |
| P1331_WT1-4 | 99 | | | | |
| VPGVAPTLVRSASETSEKRPF | | | | | |
| P-5525-LAGE1 | | | | | |
| P5525_LAGE1-1 | 100 | P5525_LAGE1-2 | 101 | P5525_LAGE1-3 | 102 |
| RLLQLHITMPFSSPMEAEL | | ITMPFSSPMEAELVRRILSR | | MEAELVRRILSRDAAPL | |
| P5525_LAGE1-4 | 103 | | | | |
| RRILSRDAAPLPRPGAVL | | | | | |
| P-P2753-MUC1 | | | | | |
| P2753_MUC1-1 | 104 | P2753_MUC1-2 | 105 | P2753_MUC1-3 | 106 |
| KKKMTPGTQSPFFLLLLLTVLTKKK | | KKKFFLLLLLTVLTVVTGSGHKKK | | KKKTVLTVVTGSGHASSTPGGKKK | |
| P-P3698-MUC1 | | | | | |
| P3698_MUC1-1 | 107 | P3698_MUC1-2 | 108 | P3698_MUC1-3 | 109 |
| PALGSTAPPVHNVTSASGSA | | PPVHNVTSASGSASGSAS | | SASGSASGSASTLVHNGT | |
| P3698_MUC1-4 | 110 | P3698_MUC1-5 | 111 | | |
| GSASTLVHNGTSARATTT | | TLVHNGTSARATTTPASK | | | |
| P-P3776-MUC-1 | | | | | |
| P3776-MUC-1_1 | 112 | P3776-MUC-1_2 | 113 | P3776-MUC-1_3 | 114 |
| TSSNHSTSPQLSTGVSFF | | TSPQLSTGVSFFFLSFHI | | TGVSFFFLSFHISNLQFNS | |
| P3776-MUC-1_4 | 115 | | | | |
| LSFHISNLQFNSSLEDPST | | | | | |
| P-P3825-MUC-1 | | | | | |
| P3825-MUC-1_1 | 116 | P3825-MUC-1_2 | 117 | P3825-MUC-1_3 | 118 |
| EMFLQIYKQGGFLGLSNI | | YKQGGFLGLSNIKFRPGS | | LGLSNIKFRPGSVVVQLT | |
| P3825-MUC-1_4 | 119 | P3825-MUC-1_5 | 120 | | |
| KFRPGSVVVQLTLAFREG | | VVVQLTLAFREGTINVH | | | |
| P-P750-NYESO | | | | | |
| P750-NYESO-1 | 121 | P750-NYESO-2 | 122 | P750-NYESO-3 | 123 |
| PDGPGGNAGGPGEAGATG | | AGGPGEAGATGGRGPRGA | | GATGGRGPRGAGAARASG | |
| P750-NYESO-4 | 124 | | | | |
| PRGAGAARASGPGGGAP | | | | | |
| P-P805-NYESO | | | | | |
| P805-NY-ESO-1_1 | 125 | P809-NY-ESO-1_2 | 126 | P805-NY-ESO-1_3 | 127 |
| RGPESRLLEFYLAMPFAT | | LLEFYLAMPFATPMEAELKKK | | AMPFATPMEAELARRSLAQD | |
| P805-NY-ESO-1_4 | 128 | | | | |
| EAELARRSLAQDAPPLPVPG | | | | | |
| P-P830-NYESO | | | | | |
| P830_NY_ESO1-1 | 129 | P380_NY_ESO1-2 | 130 | P380_NY_ESO1-3 | 131 |
| LARRSLAQDAPPLPVPGVL | | DAPPLPVPGVLLKEFTVS | | PGVLLKEFTVSGNILTIR | |
| P830_NY_ESO1-4 | 132 | | | | |
| EFTVSGNILTIRLTAADHRQ | | | | | |
| P-P5232-MAGE1 | | | | | |
| P5232_MAGE1-1 | 133 | P5232_MAGE1-2 | 134 | P5232_MAGE1-3 | 135 |
| SILESLFRAVITKKVADLVGF | | ITKKVADLVGFLLLKYRA | | DLVGFLLLKYRAREPVTK | |
| P5232_MAGE1-4 | 136 | | | | |
| LKYRAREPVTKAEMLESVIK | | | | | |
| P-P5400-MAGE1 | | | | | |
| P5400-MAGE1_1 | 137 | P5400-MAGE1_2 | 138 | P5400-MAGE1_3 | 139 |
| PARYEFLWGPRALAETSY | | LWGPRALAETSYVKVLEY | | LAETSYVKVLEYVIKVSA | |
| P5400-MAGE1_4 | 140 | P5400-MAGE1_5 | 141 | | |
| VKVLEYVIKVSARVRFFF | | VIKVSARVRFFFPSLREA | | | |
| P-P1575-HER | | | | | |
| P1575-HER_1 | 142 | P1575-HER_2 | 143 | P1575-HER_3 | 144 |
| VQGYVLIAHNQVRQVP | | VLIAHNQVRQVPLQRLRI | | VRQVPLQRLRIVRGTQLF | |
| P1575-HER_4 | 145 | | | | |
| RLRIVRGTQLFEDNYALALVL | | | | | |
| P-P2238-HER | | | | | |
| P2238-HER_1 | 146 | P2238-HER_2 | 147 | P2238-HER_3 | 148 |
| GENVKIPVAIKVLRENTS | | PVAIKVLRENTSPKANKE | | LRENTSPKANKEILDEAY | |
| P2238-HER_4 | 149 | P2238-HER_5 | 150 | | |
| PKANKEILDEAYVMAGVG | | ILDEAYVMAGVGSPYVSRKKK | | | |
| P-P103-P53 | | | | | |
| P103-P53_1 | 151 | P103-P53_2 | 152 | P103-P53_3 | 153 |
| APSWPLSSSVPSQKTYQG | | SSSVPSQKTYQGSYGFRL | | | |
| P103-P53_4 | 154 | | | | |
| YGFRLGFLHSGTAKSVTCT | | | | | |
| P-P-HAGE | | | | | |
| P131-HAGE_1 | 155 | P313-HAGE_2 | 156 | P131-HAGE_3 | 157 |
| AQTGTGKTLCYLMPGFIH | | KTLCYLMPGFIHLVLQPSKKK | | MPGFIHLVLQPSLKGQRN | |
| P131-HAGE_4 | 158 | | | | |
| LVLQPSLKGQRNRPGMLV | | | | | |

1. A pharmaceutical composition comprising at least two peptides of from 20 to 60 amino acids in length, selected from peptides comprising a sequence of at least 20 contiguous amino acids of a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48 or of a sequence having at least 80% identity to a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48, wherein each peptide comprises at least one CD8+ T-cell epitope and/or at least one CD4+ T-cell epitope.

2. The composition of claim 1, which comprises at least one peptide comprising at least 30 amino acids of a sequence shown in any one of SEQ ID NOs: 1 to 40 and 48.

3. The composition of claim 1 or 2, which comprises at least one peptide comprising at least one CD8+ T-cell epitope and at least one CD4+ T-cell epitope.

4. The composition of any one of claims 1 to 3, which comprises a peptide that has a HLA Class II allele population coverage of: at least 60% in at least 7 geographical areas; at least 60% in at least 6 geographical areas; at least 80% in at least 5 geographical areas, at least 90% in at least 2 geographical areas and/or at least 95% in at least one geographical area.

5. The composition of any one of the preceding claims, which comprises a peptide that has a HLA Class I allele population coverage of: at least 25% in at least 5 geographical areas; at least 30 in at least 2 geographical areas; and/or at least 60% in at least 1 geographical area.

6. The composition of any one of the preceding claims, which comprises at least one peptide that induces a specific T cell response in a healthy subject and/or a cancer patient, preferably wherein said peptide is capable of inducing a specific T cell response in a cancer patient which is of reduced magnitude compared to the specific T cell response to the same peptide in an age-matched healthy subject.

7. The composition of any one of the preceding claims, wherein at least one of the peptides comprises a sequence shown in any one of SEQ ID NOs: 1 to 40 and 48, or a sequence having at least 80% identity to the whole length of one of the sequences shown in SEQ ID NOs: 1 to 40 and 48.

8. The composition of any one of the preceding claims, wherein at least one peptide further comprises one or more additional amino acid at the N-terminus and/or C-terminus to increase the net positive charge and/or to reduce hydrophobicity of the peptide.

9. The composition of any one of the preceding claims, wherein the composition comprises peptides derived from at least two tumour antigens selected from MAGE3, MUC1, hTERT, MAGE1, P53, NY-ESO1, HER2/NEU, HAGE, Survivin, WTI and LAGE1.

10. The composition of any one of the preceding claims, wherein the composition comprises peptides derived from at least two of HAGE, MAGE-3, LAGE, NY-ESO-1 and MAGE-1, or from at least two of MAGE3, MUC1, hTERT and MAGE1.

11. The composition of any one of claims 1 to 10, which comprises at least one peptide selected from at least two of the following groups:
(xi) peptides comprising the sequence shown in SEQ ID NO: 40;
(x) peptides comprising the sequence shown in SEQ ID NO: 39;
(i) peptides comprising the sequence shown in any one of SEQ ID NOs: 1 to 3 and 48;
(vi) peptides comprising the sequence shown in any one of SEQ ID NOs: 26 to 29; and
(iv) peptides comprising the sequence shown in any one of SEQ ID NOs: 22 to 23;

12. The composition of claim 11, which comprises at least one peptide selected from at least three, at least four or all five of groups (xi), (x), (i), (vi) and (iv).

13. The composition of any one of claims 1 to 10, which comprises at least one peptide selected from at least two of the following groups:
(i) peptides comprising the sequence shown in any one of SEQ ID NOs: 1 to 3 and 48;
(ii) peptides comprising the sequence shown in any one of SEQ ID NOs: 4 to 7;
(iii) peptides comprising the sequence shown in any one of SEQ ID NOs: 8 to 20; and
(iv) peptides comprising the sequence shown in any one of SEQ ID NOs: 21 to 22.

14. The composition of claim 13, which comprises at least one peptide selected from at least three or all four of groups (i) to (iv).

15. The composition of claim 12, comprising:
(a) a peptide which consists of the sequence of SEQ ID NO: 40, or a peptide which is 20 to 60 amino acids in length and comprises a sequence of at least 20 contiguous amino acids of SEQ ID NO: 40;
(b) a peptide which consists of the sequence of SEQ ID NO: 39, or a peptide which is 20 to 60 amino acids in length and comprises a sequence of at least 20 contiguous amino acids of SEQ ID NO: 39;
(c) a peptide which consists of the sequence of SEQ ID NO: 29, or a peptide which is 20 to 60 amino acids in length and comprises a sequence of at least 20 contiguous amino acids of SEQ ID NO: 29;
(d) a peptide which consists of the sequence of SEQ ID NO: 23, or a peptide which is 20 to 60 amino acids in length and comprises a sequence of at least 20 contiguous amino acids of SEQ ID NO: 23; and
(e) a peptide which consists of the sequence of SEQ ID NO: 2, or a peptide which is 20 to 60 amino acids in length and comprises a sequence of at least 20 contiguous amino acids of SEQ ID NO: 2.

16. The composition of claim 15, further comprising any one, two, three, four or five peptides independently selected from:
(f) a peptide which consists of the sequence of SEQ ID NO: 28, or which is 20 to 60 amino acids in length and comprises a sequence of at least 20 contiguous amino acids of SEQ ID NO: 28;
(g) a peptide which consists of the sequence of SEQ ID NO: 22, or which is 20 to 60 amino acids in length and comprises a sequence of at least 20 contiguous amino acids of SEQ ID NO: 22;
(h) a peptide which consists of the sequence of SEQ ID NO: 24, or which is 20 to 60 amino acids in length and comprises a sequence of at least 20 contiguous amino acids of SEQ ID NO: 24;
(i) a peptide which consists of the sequence of SEQ ID NO: 18, or which is 20 to 60 amino acids in length and comprises a sequence of at least 20 contiguous amino acids of SEQ ID NO: 18; and
(j) a peptide which consists of the sequence of SEQ ID NO: 12, or which is 20 to 60 amino acids in length and comprises a sequence of at least 20 contiguous amino acids of SEQ ID NO: 12.

17. The composition of claim 16, which comprises at least one peptide independently selected from each of options (a) to (g).

18. The composition of claim 16, which comprises at least one peptide independently selected from each of options (a) to (j).

19. The composition of any one of claims 16 to 18, in which the selected peptides of options (a) to (j) are the only active ingredients or the only peptide active ingredients in the composition.

20. The composition of any one of claims 1 to 10, which comprises at least one peptide comprising or consisting of a sequence as shown in Table A1.

21. The composition of claim 20, which comprises 2, 3, 4, 5, 6, 7, 8, 9, 10, 11, 12, 13 or 14 said peptides, and optionally at least one peptide comprising or consisting of a sequence as shown in Table A2.

22. The composition of any one of the preceding claims, wherein the peptides are linked to a fluorocarbon vector.

23. The composition of any one of the preceding claims, which further comprises an adjuvant.

24. The composition of any one of the preceding claims for use in the treatment or prevention of cancer.

25. The composition of claim 24 for use in the treatment of non-small-cell lung cancer, breast cancer, hepatic cancer, brain cancer, stomach cancer, pancreatic cancer, kidney cancer, ovarian cancer, myeloma cancer, acute myelogenous leukaemia, chronic myelogenous leukaemia, head and neck cancer, colorectal cancer, renal cancer, oesophageal cancer, melanoma skin cancer and prostate cancer.

26. The composition of claim 24 or 25 for use in the treatment of non-small cell lung cancer.

27. A peptide of from 20 to 60 amino acids in length comprising at least 20 contiguous amino acids of a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48 or of a sequence having at least 80% identity to a sequence shown in any one of SEQ ID NOs: 40, 39, 29, 23, 2, 28, 22, 24, 18, 12, 8, 17, 3, 11, 1, 4 to 7, 9, 10, 13 to 16, 19 to 21, 25 to 27, 30 to 38 and 48, which peptide comprises at least one CD8+ T-cell epitope and at least one CD4+ T-cell epitope.

28. The peptide of claim 27, which comprises a peptide that has a HLA Class II allele population coverage of: at least 60% in at least 7 geographical areas; at least 60% in at least 6 geographical areas; at least 80% in at least 5 geographical areas, at least 90% in at least 2 geographical areas and/or at least 95% in at least one geographical area.

29. The peptide of claim 27 or 28, which comprises a peptide that has a HLA Class I allele population coverage of: at least 25% in at least 5 geographical areas; at least 30 in at least 2 geographical areas; and/or at least 60% in at least 1 geographical area.

30. The peptide of any one of claims 27 to 29, which is capable of inducing a specific T cell response in a healthy subject and/or a cancer patient.

31. The peptide of any one of claims 27 to 30, which is capable of inducing a specific T cell response in a cancer patient which is of reduced magnitude compared to the specific T cell response to the same peptide in an age-matched healthy subject.

32. The peptide of any one of claims 27 to 31, which comprises or consists of a sequence shown in any one of SEQ ID NOs: 40, 39, 29, 23, 2, 28, 22, 24, 18, 12, 8, 17, 3, 11, 1,4 to 7, 9, 10, 13 to 16, 19 to 21, 25 to 27, 30 to 38 and 48.

33. A peptide according to any one of claims 27 to 32, which is covalently linked to a fluorocarbon vector.

34. A method of treating or preventing cancer, the method comprising administering to a subject in need thereof a therapeutically effective amount of a composition according to any one of claims 1 to 23 or a therapeutically effective amount of a peptide according to any one of claims 27 to 32.

35. The method of claim 34, wherein the composition is administered in combination with an immune modifier that down-regulates T-regulatory cells; or an agent capable of blocking immune checkpoints.

36. The method of claim 35, wherein the immune modifier is cyclophosphamide; or the agent is a cytokine or monoclonal antibody.

37. The method of claim 36, wherein the monoclonal antibody is an anti-PD1 or anti-CTLA-4 antibody.

38. Use of a composition according to any one of claims 1 to 23, or of a peptide according to any one of claims 27 to 32, in the manufacture of a medicament for the treatment or prevention of cancer.
